SEQUENCE LISTING (8 sequences)

SEQ ID NO: 1 - DNA, 3219 bp, Arabidopsis thaliana
  CDS (1157)..(1329)
  CDS (1461)..(1590)
  CDS (1659)..(1796)
  CDS (1986)..(2318)
  CDS (2399)..(2582)
  CDS (2655)..(2824)
gttgcttgtcgaagatggtgatctctgcccgagacgcattgttggagagcctgtggacgaaatgcgcagaagagctaggagtgacattgccaacgatggttaaaagtgttaatatagaggaaaagagctaagacttgtttgtgaatatagaggaaacatgtaattatggtatcgttgatgttaagtaataacaaatgcagataaaaatgagactttaaaacgcttcaaatattgataactctctcttttcaatcggtgctaagctataactacattttcaagttagtcagaattgtgctttagttcatcttagttgataatggataagtgatgggtttgcttgaggcccattgtaaatcactagaccaaaaaaaatcagtgttattttcatggttacatgatcaatcaatcaaagcaaaccagatattctagctattaataataacaaatgcaccaaacttcaacatggttatataagttgtttaaccatgattttttaaaaaaaatcaaaatgatgtcattttatcttatacataaaccttttttcactaatggttttggaatcaaaggtcagttttatggtcaaaaacaatttggccgcccattgacttgtacaccaccaatataccaagtcgtacttaaattcttattggagaaaagttgtcatctagattcttttaaactttataatatgatatcaagaacattcctacatgaagtcattaacttgttccattctacaaacttcatcaattacattttttcggcaaaagtcataaccatatctaatctctacttcgtgggataacgcgatttagtagaagcaaactccagtaaacaaaacttaaccaattgactaaaaacacttacccgttcctcaattacttagagacacaatcatcggatatacccaactcgataccataaacaaaattgaaaattgacattacaaagaaaaagaacaaaaaccaaagaagaagacgcaaacccggtttaagggtcacgggtgactcgagaggagacttcctctttaccgttcaataaaaaaaaaagggtaaaggtagagactttataacgaaatgtcaacacccatcttctcgattcctcaatttctctagggttcttcgttttcgttgttgtgaggaattttgctccgatgtcggcggagagttgtttcggaagctcgggtgatcaaagcagcagcaaaggagtggctactcatggtggtagctatgttcagtataatgtctatggcaatctcttcgaagtttctagaaagtatgttcctcctcttcgtcctatcggtagaggcgcttatggaattgtctggtgagttccaaaattaatcagattgggtttttgaaacatgtaatgttcacttctggggttcttagatttgggaattagggttttggaatctgttgttattataataacttgtgttgataattgttgatcagtgctgctacaaactcagagactggagaggaggtagctatcaagaagattggtaatgcttttgacaatattatcgatgctaaaaggactttgagagagattaagcttctcaagcatatggatcatgaaaatgtacgtgttactaatcatcagcatctctggttttttttgtgtgaatcttatattggtttattgtgcaggtgattgctgtaaaggatataataaaaccaccgcagagagagaacttcaatgatgtttacattgtttatgagcttatggacactgatctccaccaaattattcgctctaaccaacccttaactgatgatcattgtcgggtatgcatcaatctccatcttgcttatatctagcagctgaatcttctgtttctactttagaagcagctgttattgtgttagagcatctgatgagtattgcttgatactttataagtgagagctaataaggatttcgtttgttatgctttgaagttgtgtttagtaattagtttcgattatgttttgcagttttttctgtatcagttgttgcgtgggctcaaatatgttcattcagctaatgtattgcatcgagatttgaaacctagcaatttgctcctgaatgcaaattgtgatctaaagcttggggatttcgggcttgcgaggaccaaatccgagactgactttatgactgaatatgttgttacacgttggtatcgagctccagagctgctacttaattgctctgaatacacagcagcaattgatatttggtctgtcggttgtatactcggtgaaacaatgacacgagagcccttgtttcccggtaaagattatgttcatcagcttagactcatcactgaggtaatctagtttagtatgtttcatccaaagaatcaccacttagtctctcttcacggtattaatgaatctttggaaagcagcttataggatctcctgatgactcaagcttgggattcttaagaagtgacaatgcaagaagatacgttagacagcttccacagtaccctagacagaactttgctgctagattcccaaacatgtcggctggtgcagtcgatttgctggagaaaatgcttgtttttgatccaagcagacgcatcacaggtgagttatattttatataactgcttctctgtttcttcggctattttctgaacatgttttttgtgtgtgtagttgatgaggcgttgtgccacccatatttggcgccgctgcatgatatcaacgaagaaccggtatgtgtgaggccgttcaattttgatttcgagcaacctactttgacagaagagaacattaaggagcttatataccgtgaaacagtcaagttcaatcctcaagactcagtgtgagagaaagagagagagatatatatccttaaaaggaaactcatcaaaatgctatgttttgttttgctctgcttttgttatcaaatctctgataagtttgataatatgtctgtaatttgaataacatcctaaaaaattggaaaaaaagaaaagaacaacctttgagctgtgttttgcaatagagtgtatttaaatttccttactttttagtcgaaagaaaatacagagaagtcttaactctaattacatccactaattatcttaatgatggggttgaggtccgaaggtgcgtaaccatctgttgaagcggtccataagtgacaactctatcttcttcgtcggaacattggggacaggtacggctttggtgagtggtccgaccacgactcgtagct

SEQ ID NO: 2 - protein, 376 aa, Arabidopsis thaliana
MSAESCFGSSGDQSSSKGVATHGGSYVQYNVYGNLFEVSRKYVPPLRPIGRGAYGIVCAATNSETGEEVAIKKIGNAFDNIIDAKRTLREIKLLKHMDHENVIAVKDIIKPPQRENFNDVYIVYELMDTDLHQIIRSNQPLTDDHCRFFLYQLLRGLKYVHSANVLHRDLKPSNLLLNANCDLKLGDFGLARTKSETDFMTEYVVTRWYRAPELLLNCSEYTAAIDIWSVGCILGETMTREPLFPGKDYVHQLRLITELIGSPDDSSLGFLRSDNARRYVRQLPQYPRQNFAARFPNMSAGAVDLLEKMLVFDPSRRITVDEALCHPYLAPLHDINEEPVCVRPFNFDFEQPTLTEENIKELIYRETVKFNPQDSV

SEQ ID NO: 3 - DNA, 23 bp, Artificial Sequence
  Primer
cagaccaactggtaatggtagcg

SEQ ID NO: 4 - DNA, 20 bp, Artificial Sequence
  Primer
ctgcatcggcgaactgatcg

SEQ ID NO: 5 - DNA, 22 bp, Arabidopsis thaliana
gtggtgataattgttgatcagt

SEQ ID NO: 6 - DNA, 30 bp, Arabidopsis thaliana
gtggtgataatttgataattgttgatcagt

SEQ ID NO: 7 - DNA, 29 bp, Arabidopsis thaliana
gtggtgataaatgataattgttgatcagt

SEQ ID NO: 8 - DNA, 25 bp, Artificial Sequence
  Synthetic oligonucleotide
ttttttttttttttttttttttttt

(19) United States
(12) Patent Application Publication
(10) Pub. No.: US 2004/0172685 A1
(43) Pub. Date: Sep. 2, 2004

(54) Method of using MAPK4 and orthologues thereof to control plant disease resistance and plant growth

(76) Inventors: John Mundy, Valby (DK); Anders Bogh Jensen, Humlebaek (DK); Morten Petersen, Copenhagen V (DK); Henrik Naested, Copenhagen N (DK); Peter Brodersen, Copenhagen O (DK)

Correspondence Address: FOLEY AND LARDNER, SUITE 500, 3000 K STREET NW, WASHINGTON, DC, 20007, US

(21) Appl. No.: 09/730,478

(22) Filed: Dec. 6, 2000

Related U.S. Application Data
(60) Provisional application No. 60/169,614, filed on Dec. 8, 1999.

(30) Foreign Application Priority Data
Dec. 6, 1999 (DK) PA 1999/01746

(51) Int. Cl. A01H 1/00; C12N 15/82

(52) U.S. Cl. 800/287; 435/468

(57) ABSTRACT

Uses of MAPK4, a member of the mitogen-activated protein (MAP) kinase family, are provided herein, based on the discovery that the MAPK4 negatively regulates the expression of genes associated with disease (e.g. PR-genes) and wound responses in plants such that the loss of MAPK4 function leads to their derepression. Methods are disclosed for controlling the growth of a plant and/or the expression of at least one wounding or pathogen response gene in said plant, the method comprising altering in the plant the level of the gene product of a MAPK4 gene. Furthermore, transgenic plants transformed with an MAPK4 construct and having enhanced wound and/or disease resistance are disclosed.

### FIELD OF THE INVENTION

This invention relates to the field of molecular biology and genetic transformation in higher plants. More specifically, the invention relates to novel use of genes encoding MAP kinase 4 to control the expression of plant wounding and/or pathogen response genes and to control the growth of a plant.

### TECHNICAL BACKGROUND OF THE INVENTION

Bacterial, fungal, insect and viral infections of plants grown for food and fiber cause substantial economic losses to farmers and consumers. The use of resistant cultivars is the most economical and effective method of controlling diseases. With the advent of transgenic plant technology, it is possible to identify natural host defence mechanisms and to transfer genes associated with these mechanisms to susceptive plants or to control expression of such genes in commercial cultivars.

Plants are exposed to a variety of environmental stresses including wounding caused by mechanical injury or attacks by potential pathogens. The survival of plants in this environment is only possible with sophisticated defence strategies. Plant disease resistance is elicited by specific recognition of pathogen-derived molecules (Staskawicz et al., 1995). These interactions lead to rapid necrosis at the site of pathogen entry (the hypersensitive response, HR) and induction of a plant immune response, known as systemic acquired resistance (SAR; Yang et al., 1997). SAR provides protection in uninfected parts of the plant against a spectrum of pathogens and is correlated with the expression of pathogenesis-related (PR) proteins, some with antimicrobial activity. Some of these genes are only induced at the site of attack, others are expressed throughout the plant and thus protect it from attack at distant sites. The onset of SAR is also associated with increased levels of salicylic acid (SA) both at the infection site and systemically (Malamy et al., 1990). SA is necessary and sufficient for SAR induction since exogenous SA application induces SAR and PR gene expression (Ward et al., 1991), while expression in plants of the bacterial salicylate hydroxylase (NahG), depletes SA and suppresses SAR (Gaffney et al, 1993).

Genetic approaches have been used in Arabidopsis to unravel plant defense pathways. Screens have identified recessive mutants affected in SA signalling that are also hyper-susceptible to pathogens. For example, the pad4, sid1 and sid2 mutations compromise SA accumulation in response to pathogen infection (Zhou et al., 1998; Nawrath and Metraux, 1999). The eds1 mutation also operates upstream of SA-mediated plant defenses (Falk et al., 1999). In contrast, npr1 mutants are able to accumulate SA but fail to mount SAR after pathogen infection or application of SA, implicating NPR1 in SA perception and downstream responses (Cao et al., 1994; Delaney et al., 1995). PAD4 and EDS1 encode lipase-like proteins (Falk et al., 1999; Jirage et al., 1999), whereas NPR1 encodes an ankyrin repeat protein (Cao et al., 1997). NPR1 interacts with basic leucine zipper transcription factors that bind to PR1 promoter elements, suggesting a direct link between NPR1 activity and regulation of PR gene expression (Zhang et al., 1999).

Other Arabidopsis mutations cause enhanced disease resistance. While many of these mutants exhibit HR-like lesions in the absence of pathogen challenge, so called lesion-mimic mutants, there are only a few reports of constitutive defense mutants without necrotic lesions (cpr1, Bowling et al. (1994); cpr6, Clarke et al., 1998). Most if not all constitutive defense mutants accumulate elevated levels of SA and express PR genes constitutively. The presence of nahG in these mutants suppresses PR gene expression and distinct aspects of their enhanced resistance to bacteria and oomycete pathogens.

While these genetic analyses confirm the importance of SA and NPR1 in regulating SAR, they also reinforce evidence for both NPR1- and SA-independent disease resistance pathways that are regulated by ethylene and jasmonic acid (JA; Pieterse and van Loon, 1999). For example, PR gene expression in cpr6 requires SA but not NPR1, although NPR1 is necessary for bacterial resistance (Clarke et al., 1998). In contrast, the ssi1 mutation completely bypasses npr1 but depends on SA to induce both PR1 and expression of PDF1.2, a JA-responsive defensin (Shah et al., 1999). Thus, the CPR6 and SSI1 proteins may participate in signal communication between SA- and JA-dependent pathways. Such pathway crosstalk is consistent with studies demonstrating antagonism between SA and JA signalling in defenses against pathogens and insect herbivores (Felton et al., 1999).

Mitogen-activated protein kinases (MAPKs or MPKs) have been identified in plants which are thought to be involved in the defence response to wounding and pathogen attacks (Mizoguchi et al., 1997). MAP kinases such as Arabidopsis MPK4 (AtMPK4) act downstream of MAPK kinases (MAPKK) and MAPKK kinases (MAPKKK) in reversible protein phosphorylation cascades to mediate intracellular signals. Activation of the MAP kinases occurs through threonine/tyrosine phosphorylation catalysed by the dual-specificity MAPKK, which in turn is activated through serine phosphorylation catalysed by MAPKKK. While these cascades amplify specific signals, they may integrate different signals by crosstalk via higher-order complexes (Madhani & Fink, 1998). Arabidopsis contains numerous MAPKKs and MAPKs although the signals their pathways transduce remain unknown (Mizoguchi et al., 1997). As an example, yeast 2-hybrid experiments indicate that Arabidopsis AtMAPK4 and the MAPKKs AtMKK2 and AtMEK1 specifically interact with the MAPKKK AtMEKK1 (Ichimura et al., 1998). They may therefore participate in one or more cascades. Morris et al. (1997) showed that AtMEK1 mRNA accumulates slowly after wounding, while AtMEKK1 mRNA accumulates in response to cold, salinity and touch (Mizoguchi et al., 1996).

An explanation for the apparent induction of expression of these MAPKK and MAPKKK genes is that the kinases are acting downstream of them and are part of a kinase cascade mediating responses to these stress stimuli (Ichimura et al., 1998). A model explaining their stress-induced expression is that the kinase cascade positively regulates responses to the stress, and their own expression is part of a positive feedback or amplification loop. In this model, the activated MAPK phosphorylates downstream targets such as transcription factors, which activate or derepress the expression of genes needed for stress tolerance or adaptation.

Biochemical work on MAPKs in other plant systems have identified such MAPKs which are phosphorylated and activated by wounding, pathogenic inoculation, pathogen elicitors and salicylate (Romeis et al. 1999; Ligterink et al. 1997; Zhang & Klessig 1998). The 48 kD MAP kinase, ERMK, is rapidly activated upon high-affinity binding of a fungal elicitor to a plasma membrane receptor in parsley cells (Ligterink et al. 1997). The activated ERMK is translocated into the nucleus where it may be involved in the transcriptional activation of defence genes.

A MAP kinase SA-induced-protein-kinase, termed SIPK, was first identified to be activated in tobacco cells by SA treatment and involved in several plant defence response (Zhang & Klessig 1997). Later it was shown that the MAP kinase, encoded by the WIPK gene, was activated by various fungal elicitors and wounding (Zhang & Klessig 1998). Thus, in WO 99/43796 is disclosed the WIPK gene and its use in the development or enhancement of resistance to microbial pathogens in plants.

However, it is also possible that these or other MAPKs may negatively regulate the expression of wound or pathogen responsive genes. For example, Hua & Meyerowitz (1998) demonstrated that a loss-of-function mutation of the ethylene receptor-related genes and the Arabidopsis MAPKKK CTR1 resulted in constitutive ethylene responses, revealing that the these proteins negatively regulate ethylene responses. Furthermore, Bardwell et al. (1998) describes that the non-phosphorylated, inactive yeast MAPK Kss1 acts as a repressor, while pathway stimulation leads to Kss1 phosphorylation and loss of repression.

In addition to the controlling of wounding and pathogens response genes, MAP kinases may be involved in the regulation of cellular and developmental events in a plant. For example, dwarfism and sterility in MAP kinase mutants may be due to metabolic imbalance resulting from the energy required for mounting a constitutive pathogen response. Other mutants with lesions in such stress response pathways also exhibit growth defects including dwarfism. For example, dwarf plants lacking the CTR1 MAPKKK and upstream ethylene receptors exhibit constitutive responses to the hormone ethylene (Hua & Meyerowitz, 1998). Similarly, constitutive pathogen response (CPR) mutants constitutively express pathogenesis related (PR) genes and are semi-dwarfed (Bowling et al., 1994). This study suggest that in CPR mutants salicylate may be involved as a signal controlling systemic acquired resistance (SAR).

Thus, the above-mentioned studies suggest that MAP kinases are an important component in the signal transduction pathways of plant defence to pathogen infection and wounding and in the regulation of plant growth. Genes encoding these MAP kinases can thus be used in a variety of ways to improve or enhance the disease resistance response in commercially important cultivars and to control the growth of such cultivars. Accordingly, there is an industrial need to identify new useful genes that are involved in such disease resistance response and growth and to determine their function and mechanism in order to be able to use them in the control of the expression of defence genes and growth in commercial cultivars.

### SUMMARY OF THE INVENTION

Accordingly, it is the primary objective of the present invention to provide methods whereby the growth and/or the plant disease resistance response of plants including commercially important cultivars is enhanced.

Thus, in a first aspect, the present invention pertains to a method of controlling the growth of a plant and/or the expression of at least one wounding or pathogen response gene in said plant, the method comprising altering in the plant the level of the gene product of a MAPK4 gene.

According to another aspect of the invention a transgenic plant having enhanced wound and/or disease resistance is provided, said plant comprising an antisense MAPK4 construct, wherein said construct leads to an increase in the expression of wounding and/or pathogen responsive genes. Furthermore, the present invention provides a transgenic plant having enhanced wound and/or disease resistance, said plant comprising a constitutively active form of MAPK4. The present invention also provides a transgenic plant having enhanced wound and/or disease resistance, said plant comprising a catalytically inactive MAPK4 construct, wherein said construct leads to an increase in the expression of wounding and/or pathogen responsive genes. In a still further aspect the present invention provides a transgenic plant having enhanced wound and/or disease resistance, said plant comprising a mutation in the MAPK4 gene which results in a loss of function of said gene, wherein said mutation leads to an increase in the expression of wounding and/or pathogen responsive genes.

In yet another aspect the present invention relates to a recombinant DNA construct comprising the coding region of MAPK4 gene operably linked in an antisense orientation to an appropriate promoter. Furthermore, the present invention provides a transgenic plant cell transformed with the DNA construct according to the invention.

In a still further aspect the present invention relates to a method for screening a plant population for plants carrying an insertion element within the MAPK4 gene whereby the gene is functionally inactivated, the method comprising the steps of a) providing a MAPK4 specific primer and an insertion element specific primer, b) providing DNA of each of said plants, c) performing PCR reactions using said primers, and d) selecting a plant carrying an insertion element within the MAPK4 gene whereby the gene is functionally inactivated by identifying a PCR product primed by said primers. Furthermore, the invention pertains to the use of a MAPK4 gene for providing MAPK4 primers useful in the method according to the invention.

The novel function of MAPK4 identified in accordance with the present invention and described in greater detail below indicates that MAPK4 may play an important role in signal transduction for activation of plant defence mechanisms against plant pathogens and wounding and in developmental events in a plant. Accordingly, the new methods, plants and plant cells provided herein offer a novel and significant advance in the field of transforming higher plants and plant breeding to enhancing the plant disease resistance response in the most important agronomic plants.

### DETAILED DISCLOSURE OF THE INVENTION

The MAPk4 gene was cloned from Arabidopsis thaliana by Mizoguchi et al. (1993) and is a member of a gene family consisting of at least nine members (AtMPK1-9) which are classified into four subgroups based on phylogenetic analysis of their amino acid sequences (Mizoguchi et al. 1997). It was shown that AtMAPK4 may act downstream of MAPKK and MAPKKK in a reversible protein phosphorylation cascade, as described above, to mediate intracellular signals. However, the specific function of AtMAPK4 in the signal transduction has until now been unknown.

As it is shown in Example 1 below, it was possible for the inventors of the present invention to identify by visual examination of stable transposant lines a recessive dwarf Arabidopsis mutant (mpk4), caused by the insertion of a modified maize Ds transposon in the MAP kinase 4 gene (Mpk4). By analysis of the nature of the mpk4 knockout, it was found that the growth of a plant and/or the expression of wounding or pathogen reponse in a plant is controlled by the level and activity of the gene product of the MAPk4 gene. As used herein, the terms “MAPK4 gene” and “MPK4 gene” is used interchangeably and designate the gene coding for the MAPK4 protein. The inventors surprisingly found that the MAPK4 in one instance negatively regulates the expression of genes associated with disease (e.g. PR-genes) and wound responses such that the loss of MAPK4 function leads to their derepression. In the present context, the expression “negatively regulates” indicates the genes' capability to suppress or depress the expression of specific genes. Furthermore, it was found that the expression of the MAPK4 gene is required for responses to jasmonates. As used herein, the term “jasmonates” relates to the whole family of jasmonates and include e.g. jasmonate (JA) and methyl jasmonate (MeJA).

Based on this unexpected finding it was possible to provide the above method to control the growth of a plant and/or the expression of at least one wounding or pathogen response gene in a plant by using the gene encoding the Arabidopsis MAPK4 and orthologues from other plant species. The term “orthologues” is used interchangeably with the term “homologues” and relates to genes and their encoded proteins of similar function but occurring in different plant species. Hybridisation techniques e.g. described in Maniatis et al. (1982) may be used to identify such orthologues of AtMAPK4.

Although the use of the Arabidopsis thaliana MAPK4 cDNA (AtMPK4) is exemplified herein, this invention is intended to encompass the use of MAPK4 nucleic acids and MAPK4 proteins from other plant species that are sufficiently similar to be used instead of the Arabidopsis MAPK4 for the purposes described below. As described above, MAP kinases are found in a variety of plant species and are highly conserved among plants (Mitzoguchi et al. 1996). However, a person skilled in the art would expect to find sequence variation among these MAP kinases, due to small changes in the genetic code, while still conserving the unique properties of the MAPK4 gene intended for the use in the present invention. Thus, the present invention contemplates the use of MAPK4 nucleic acid molecules that encode MAPK4 polypeptides, the nucleic acid molecules having a homology of at least 70%, preferably at least 80% and most preferably at least 90% homology, such as at least 95% homology, with the nucleotide sequence of Arabidopsis MAPK4 shown in SEQ NO ID:1. In another aspect, the invention relates to MAPK4 polypeptides that include an amino acid sequence having at least 50%, e.g. at least 60%, including at least 70%, such as at least 80%, e.g. at least 90% homology to the sequence shown in SEQ NO ID:2.

In the present context, the term “homology” indicates a quantitative measure of the degree of homology between two amino acid sequences of equal length or between two nucleotide sequences of equal length. If the two sequences to be compared are not of equal length, they must be aligned to the best possible fit. The sequence identity can be calculated as

$$\frac{( {N}_{ref} - {N}_{dif} ) 100}{{N}_{ref}} ,$$

wherein Ndif is the total number of non-identical residues in the two sequences when aligned, and wherein Nref is the number of residues in one of the sequences. Hence, the DNA sequence AGTCAGTC will have a sequence identity of 75% with the sequence AATCAATC (Ndif=2 and Nref=8). A gap is counted as non-identity of the specific residue(s), i.e. the DNA sequence AGTGTC will have a sequence identity of 75% with the DNA sequence AGTCAGTC (Ndif=2 and Nref=8). Sequence identity can alternatively be calculated by the BLAST program, e.g. the BLASTP program (Pearson & Lipman (1988) (www.ncbi.nlm.nih.gov/cgi-bin/BLAST). In one aspect of the invention, alignment is performed with the global align algorithm with default parameters as described by Huang & Miller (1991), available at http://www.ch.embnet.org/software/LALIGN_form.html.

The MAPK4 orthologues according to the present invention include, but are not limited to, nucleic acid sequences similar to the AtMPK4 sequence but differing from this sequence by one or more substitutions, deletions and/or additions.

In one preferred embodiment of the method according to the invention the level of the gene product of a MAPK4 gene is altered in the plant by the following steps a) providing a recombinant DNA construct in a suitable vector in which the coding region of a MAPK4 gene is operably linked in an anti-sense orientation to an appropriate promoter such that the expression of the MAPK4 gene is regulated by said promoter; b) transforming regenerable cells of a plant with said recombinant DNA construct; and c) regenerating a transgenic plant from said transformed cell.

As used herein, the term “transgenic plant” refers to a plant which by the process of transformation is made to contain DNA sequences which are not normally present in the plant, or DNA sequences which are in addition to the sequences which are normally in the plant, or DNA sequences which are normally in the plant but which are altered compared to the native sequence.

The term “DNA construct” refers to a genetic sequence used to transform plant cells and generate progeny transgenic plants. A DNA construct comprises at least a coding region for a desired gene product, operably linked in an anti-sense orientation to the 5′ and 3′ regulatory sequences for the expression in plants. In preferred embodiments, such DNA constructs are chimeric, i.e. consisting of a mixture of sequences from different sources. However, non-chimeric DNA constructs may also be used in the present invention. Furthermore, the DNA construct may also be operably linked in a sense orientation, according to the discussion below.

The term “anti-sense” refers to the sequence of the DNA strand that is complementary to the sequence of the sense strand and cannot be translated into the polypeptide encoded by the structural gene. For purposes of the present invention, antisense refers to a DNA construct that is operably linked to a promoter in the reverse orientation such that when the DNA is transcribed, an antisense RNA molecule is produced that has a nucleotide sequence that is complementary to and capable of hybridising to an mRNA produced from the same DNA sequence in the sense orientation leading to a reduced level of endogenous MAPK4 protein. The term “sense” as used herein, refers to the sequence of the DNA strand of a structural gene that is transcribed into an mRNA molecule copy which is then translated into the polypeptide encoded by the structural gene. The use of a DNA construct in a vector in which the coding region of an intact MAPK4 is operably linked in a sense orientation to a promoter is discussed below.

The term “operably linked” means that the regulatory sequences which are necessary for the expression of the coding sequence are placed in the DNA molecule in the appropriate position relative to the coding sequence so as to effect the expression of the coding sequence. The term “promoter” means herein a DNA sequence which causes transcription of DNA into an RNA molecule. For purposes herein, promoter is used to denote DNA sequences that permit transcription in a plant. Furthermore, the term “vector” means a DNA molecule that is capable of replicating in a cell to which another DNA sequence can be operably linked as to bring about replication of the attached DNA sequence. Commonly used vectors are discussed below and include bacterial plasmids and bacteriophages.

The DNA constructs can be incorporated in plant cells using conventional recombinant DNA technologies. Generally, such techniques involve inserting the DNA in an expression vector which contains the necessary elements for the transcription and translation of the inserted protein coding sequence and one or more marker sequences to facilitate selection of transformed cells or plants. Once the DNA construct (chimeric or non-chimeric) has been cloned into an expression vector, it may be introduced into the plant cell using conventional transformation procedure known by a person skilled in the art. These include, but are not limited to, Agrobacterium vectors, PEG treatment of protoplast, biolistic DNA delivery, bombardment with gold particles coated with the DNA construct, electroporation of protoplast or direct DNA uptake as generally described in Plant Tissue Culture Manual (Lindsey, 1992, Kluwer Academic Pubs., Dordrecht). However, the method of transformation depends upon the plant to be transformed.

The term “plant cell” is intended to encompass any cell derived from the plant including undifferentiated tissues such as callus and suspension cultures, as well as plant seed, pollen or plant embryos. Plant tissues suitable for transformation includes leaf tissue, root tissue, meristems, protoplasts, hypocotyls, cotyledons, scutellum, shoot apex, root, immature embryo, pollen and anther.

In the present context, the term “resistance” indicates the ability of a transgenic plant cell to resist the effect of wounding and pathogen attack. By “enhanced” or “increased” resistance is meant a greater level of resistance to a disease-causing pathogen and/or wounding in a transgenic plant (or cell or seed hereof produced in the method of the invention than the level of resistance relative to a control plant (e.g. a non-transgenic plant or non-transgenic mother plant). In preferred embodiments, the level of resistance to wounding or to a pathogen is at least 10%, e.g. at least 20%, including at least 30%, such as at least 40%, e.g. at least 50% greater than the resistance of a control plant. In other preferred embodiments, the level of resistance to wounding and/or a pathogen is at least 60%, e.g. at least 70%, including at least 80%, such as at least 90% greater than the resistance of the control plant; with up to 100% above the level of resistance as compared to the control plant being most preferred. The level of resistance is measured by conventional methods. For example, the level of resistance to wounding or a pathogen may be determined by comparing physical features and characteristics (e.g. plant height and weight, or by comparing disease symptoms, e.g. delayed lesion development, reduced lesion size, leaf wilting and curling, water-soaked spot, and discoloration of cells) of transgenic plants (Jach et al., 1995, Whalen et al., 1991).

As used herein, the term “pathogen” relates to an organism whose infection of viable plant tissue elicits a disease response in the plant tissue. In one preferred embodiment of the method according to the invention the transgenic plant has an enhanced resistance to plant pathogens selected from the group consisting of virus, viroids, fungi, bacteria, insects, mycoplasma, and nematodes. Plant diseases caused by these pathogens are described in the Chapters 11-16 of Agrios (1988).

The term “disease defence response” is used interchangeably with the terms “disease resistance response” or “pathogen response” and refers to a change in metabolism biosynthetic activity or gene expression that enhances the plants' ability to suppress the replication and spread of a microbial pathogen (i.e. to resist the microbial pathogen). Examples of plant disease defence reponse include, but are not limited to, syntheses of antimicrobial compounds (referred to as phytoalexins) and induction of expression of defence genes, whose products include e.g. peroxidases, cell wall proteins, proteinase inhibitors, hydrolytic enzymes, pathogenesis related (PR) proteins and biosynthetic enzymes. Some of these defence response pathways are salicylic acid (SA) dependent, while others are partially SA dependent and still others are SA independent (Bowling et al. 1994). Furthermore, some defence response pathways are jasmonic acid (JA) or methyl jasmonate (JA) dependent as explained in detail below.

A plant species or cultivar may be transformed with a DNA construct that encodes a polypetide from a different plant species or cultivar (e.g. rice transformed with a gene encoding the tobacco MAPK4) or alternatively, a plant species or cultivar may be transformed with a DNA construct that encodes a polypeptide from the same plant or cultivar.

In one useful embodiment of the method according to the invention the transgenic plant has an enhanced wound and/or disease resistance. Since MAPK4 and its othologues are likely to be involved in a MAP kinase signal transduction cascade that negatively regulates defence gene expression, transgenic plants with an inactive MAPK4 produced in the method according to the invention have an altered expression of their naturally plant disease defence genes such as PR-genes. If a constitutive promoter is used, antisense MAPK4 transcripts lead to reduced accumulation and/or expression of endogenous MAPK4 mRNA, thereby reducing the expression of functional MAPK4 protein. Depending upon the levels of MAPK4 antisense transcripts produced, which may be determined by the transcriptional strength of the promoter used, the reduced levels of endogenous MPK4 protein may lead to the constitutive expression of PR genes such that they are expressed in a healthy plant in the absence of wounding and/or pathogen presence or infection. The plant is thus prepared for an attack by pathogens.

As discussed below, the entire transcribed regions of an MAPK4 gene or parts thereof may be used to produce antisense MAPK4 under the control of inducible promoters. In this case, PR gene expression would be derepressed in response to the application to plants of an inducing substance or mixture or in response to a given abiotic treatment.

As described above, infection of leaves by wounding or microbial pathogens causes an increase in the endogenous level of salicylic acid (SA) followed by the induction of the expression of PR proteins locally and systematically and by the onset of systemic acquired resistance (SAR). The inventors surprisingly found, that the loss of MPK4 activity results in the accumulation of PR proteins and in the establishment of SA-dependent SAR. Thus, the inventors showed for the first time that MPK4 kinase activity is required to turn off SA-dependent SAR. This indicates that pathogen attack of a wildtype plant should lead to a decrease in Mpk4 gene expression and/or MPK4 protein levels to allow SA-dependent SAR to develop. Thus, in one preferred embodiment, the transformation with the antisense MAPK4 construct leads, relative to the wild type plant, to an increased content of salicylic acid (SA) in the transgenic plant. In a further embodiment, the increased content of salicylic acid results in the induction of a SA-dependent systemic acquired resistance (SAR).

As mentioned above, some defence response pathways are jasmonic acid (JA) or methyl jasmonate (JA) dependent. As shown in the below Example, SA-responsive genes and JA-responsive genes may work in part antagonistically in a plant to induce different types of resistance to different types of pathogens (Thomma et al., 1998; Schenk et al., 2000). However, although these two major pathways are oppositely affected in mpk4 mutants, in contrast other plants such as e.g. tobacco, a simultaneous activation of the SA and JA-dependent pathway is possible. Thus, in a useful embodiment of the method according to the invention, the DNA construct comprises a further MAPK4 gene which is operably linked to an appropriate promoter, such that the expression of that MAPK4 gene is regulated by said promoter.

By way of example, an increased JA-responsive gene expression may be obtained by the following two approaches. The first approach is a constitutive overexpression of a wildtype MPK4 protein which reduces the induction of SA-responsive SAR and makes the JA-responsive gene expression hyperinducible. Thus, in a preferred embodiment of the method according to the invention, the further MAPK4 gene is overexpressed. This increased expression of the MAPK4 gene leads to an increased response to jasmonates in the transgenic plant.

The second approach involves the production of inducible MPK4 versions either wildtype protein or constitutively activated MPK4 forms. Induction of the constitutively activated MPK4 forms will boost JA-inducible defences. By way of example, there are at least two ways in which to produce a constitutively active MPK4 form. As used herein, the term “constitutively active MPK4” relates to a kinase form which is continually or more than normally capable of phosphorylating its sepcific substrates. This can in theory be accomplished in two ways.

First, to become active, two residues in the activation loop of MAPKs (residues Thr201 and Tyr203 in MPK4) need to be phosphorylated, generally by the upstream MAPKK (Huang et al., 2000). In many protein kinases the substitution of such residues with Asp (D) or Glu (E), which are bulky, acidic residues, mimics phosphorylated Thr and Tyr, resulting in that the kinase becomes constitutively active. While this mutagenic approach does not work for MAPKs due to structural constraints, Huang et al. (2000) have shown that MPK4 may autophosphorylate on Tyr203. It is therefore possible that mutagenesis of Thr 201 to Asp (D) or Glu (E) would create a hyperactive MPK4. This would thus render the plant hyperinducibl JA-responsive.

In a second approach, a fusion between a MAPK to an upstream MAPKK, which is made catalytically constitutive active by mutation (see below) leads to a constitutively active MAPKK/MAPK fusion protein. This is due to the fact that if the MAPKK is active itself, its proximity to the MAPK allows it to phosphorylate the MAPK all the time, thereby making it constitutively active. As described above, MPK4 interacts with two Arabidopsis MAPKKs, AtMEK1 and AtMKK2. It has been shown that AtMKK2 Thr 220 and Thr 226 are required for AtMKK2 activity. Since these residues align with those mutated in other MAPKKs (Miyata et al., 1999 and Robinson et al., 1998) to produce constitutively active MAPKKs and MAPPK/MAPK fusions, it is considerably that substitution of AtMKK2 Thr 220 and Thr 226 for Asp (D) or Glu (E) would produce constitutively active AtMKK2. Thus, it is possible to make constitutively active AtMKK2 and AtMEK1. These can subsequently be fused to wild type MPK4 in either of the ways described by Miyata et al 1999 (MAPKK N-terminal to MAPK) or Robinson et al., 1998 (MAPK N-terminal to MAPKK).

Accordingly, in further preferred embodiments of the method according to the invention the gene product of the further MPK4 gene is produced in a constitutively active form. The increased activity of MAPK4 leads to an increased jasmonate (JA) response in the transgenic plant. These responses include, but are not limited to, the induction of resistance or response(s) to the fungus Alternaria bassicicola and the induction of certain defensive genes such as the gene coding for PDF1.2 (a defensin) and THI2.1 (a thionin), both of which are anti-microbial polypeptides.

However, it is also possible to produce transgenic plants wherein the JA-responsive gene expression is increased. Thus, in an interesting embodiment, the level of the gene product of a MAPK4 gene is altered in the plant by the following steps a) providing a gene coding for an active MAPKK, b) fusing said MAPKK gene with a recombinant DNA construct in a suitable vector in which the coding region of a MAPK4 gene is operably linked to an appropriate promoter such that the expression of the MAPK4 gene is regulated by said promoter, in order to obtain an activated MAPKK/MAPK4 fusion protein, c) transforming regenerable cells of a plant with said MAPKK/MAPK4 fusion protein in order to express a constitutively activated MAPK4 in said cells, d) regenerating a transgenic plant from said transformed cell.

In one useful embodiment of the present method, the expression of the MAPK4 gene is increased which in turn leads, relative to the wild type plant, to an increased response to jasmonates (JAs) in the transgenic plant. In further embodiments, the increased JA-response results in the expression of JA-responsive genes selected from the group consisting of the PDF1.2 gene and the THI2.1 gene.

In a useful embodiment of the present invention the method comprises the steps of a) providing a recombinant DNA construct in a suitable vector in which the coding region of a gene coding for a catalytically inactive MAPK4 is operably linked in a sense orientation to an appropriate promoter such that the expression of the catalytically inactive MAPK4 encoding gene is regulated by said promoter; b) transforming regenerable cells of a plant with said recombinant DNA construct; and c) regenerating a transgenic plant from said transformed cell. It will be appreciated that the DNA construct can be incorporated into plant cells using the conventional recombinant techniques as described above.

In one embodiment, this method takes advantage of the fact that it is possible to make phosphorylatable but catalytically inactive MAP kinases by mutating amino acid residues involved in ATP binding. These forms can still interact and be modified by other proteins such as by phosphorylation by one or more upstream MAPKK(s). For example, phosphorylatable, inactive MAPK4 forms could be generated by in vitro mutagenesis of residues corresponding to Arabidopsis MAPK4 tyrosine 54 or lysine 72 to phenylalanine or arginine, respectively. Such catalytically inactive MAPK forms, expressed as sense transgenes, may compete with endogenous, catalytically activatable forms either for interaction with upstream proteins, such as MAPKKs, or for interaction with downstream partners such as transcription factor(s) controlling the expression of PR genes. Depending upon the strength of this competition, a catalytically inactive form may lead to derepression, and therefore expression of PR genes.

In another embodiment of the invention the product of the MAPK4 gene cannot be phosphorylated. Non-phosphorylatable and therefore inactive MAPKs may e.g. be produced by mutating amino acid residues involved in MAPK phosphorylation by MAPKKs, for example by using in vitro mutagenesis of residues corresponding to Arabidopsis MPK4 threonine 201 and tyrosine 203 to alanine and phenylalanine, respectively. Thus, in an interesting embodiment, the product of the inactive MAPK4 gene is non-phosphorylatable. The generation of such non-phoshorylatable, catalytically inactive MAPK forms is known by the person skilled in the art. However, without limitations, one method of generating non-phosphorylatable, inactive MPK4 forms could be by using in vitro mutagenesis of residues corresponding to Arabidopsis MPK4 threonine 201 and tyrosine 203 to alanine and phenylalanine, respectively.

This embodiment of the invention has the advantage over antisense strategy that the control and monitoring of expression levels of sense transgenes, such as catalytically inactive, sense MAPK4 forms, may be more simple than the control and monitoring of expression levels of an antisense construct and of the endogenous, catalytically activatable MAP kinases. Further it should be noted that a sense form of a catalytically inactive, Arabidopsis MAPK4 might, as discussed above, exhibit derepressive effects not only in Arabidopsis but also in other species depending upon structural and functional conservation. This means that a single sense construct can be used in many different plants.

As shown in the Example below, one possible way of controlling the growth of a plant and/or the expression of at least one wounding or pathogen response gene in a plant is by mutating the MAPK4 gene in said plant in order to obtain a so-called knock-out mutant or a loss of function mutant. Thus, in one useful embodiment of the method according to the invention the level of the gene product of a MAPK4 gene is altered in the plant by the following steps a) mutating in a regenerable plant cell the MAPK4 gene so as to obtain a loss of function of said gene, and b) regenerating a transgenic plant from said transformed cell. In a further embodiment, said mutation is provided by inserting an insertion element within the MAPK4 gene, wherein the insertion element is selected from the group consisting of a T-DNA and a transposon. As used herein, the term “insertion element” relates to a segment of DNA, such as T-DNA or transposons, which is inserted in a specific gene.

In the present context, the terms “pathogenesis related (PR) genes” or “PR” genes means genes or their encoded proteins which are expressed, synthesised or activated in conjunction with the infection with a pathogen to which the plant is usually resistant. Thus, in another useful embodiment, the PR genes are expressed in association with the establishment of systemic acquired resistance (SAR) and local acquired resistance response (LAR) or a jasmonic acid or methyl jasmonate (JA) dependent reponse. In a further embodiment, the wounding and pathogen reponse genes are genes coding for gene products selected from the group consisting of chitinase, extensin (EXT1), PR1, PR5, lipid transfer protein (LTP), β-1,3-glucanase (BGL2/PR2), β-1,3-glucanase (BGL3), glutathione S-transferase (ERD11), glutathione S-transferase (PM24), ascorbate free radical reductase, lipid transfer protein, pectin methylesterase (PME1), LRR receptor kinase, oxalate oxidase-like (GLP5), osmotin, thioni, glycin-rich proteins (GPRs), phenylalanine ammonia lyase (PAL), lipoxygenase (LOX), monodehydroascorbate reductase, a lipid transfer protein (MTE17.7), a proline-rich protein, PDF1.2 and THI2.1. In a further preferred embodiment, the wounding or pathogen response gene is overexpressed. Furthermore, in a useful embodiment, the overexpression results in an enhanced resistance to plant pathogens selected from the group consisting of viruses, fungi, bacteria, insects and nematodes.

The term “wounding response” relates to a change in metabolism, biosynthetic activity or gene expression that occurs in a plant in response to wounding (e.g. cutting, abrasion). Wounding related genes and their encoded proteins are activated in association with wounding of a plant. These genes are also referred to as wounding inducible genes, as they may be induced in a disease defence response or a wounding response, with similar or differing kinetics of induction.

As mentioned above, transgenic plants produced by the method according to the invention and comprising an altered level of the gene product of a MAPK4 gene may also exhibit visible, phenotypic abnormalities including reduced size. Thus, in one useful embodiment of the method of the present invention the transgenic plant, relative to a wild type plant, has a reduced growth. Without intending to limit the invention in any way, the inventors propose that the dwarfism of plants transformed with the antisense MAPK4 construct may be due to metabolic imbalance resulting from the energy required to mount a constitutive pathogen response and may be caused by many types of genetic lesions. The responses of mpk4 mutants and wild type plants were found to be equivalent to various plant growth regulators and abiotic stresses as measured by a panel of physiological and molecular assays. As shown in the Example below, northern hybridisation demonstrated that mpk4 mutants ectopically over-expressed several pathogenesis responsive (PR) genes.

The reduction in MAPK4 protein and/or its activity may also be such that a plant produced with the method according to the invention is phenotypically different from other plants which also are resistant to wounding and/or pathogens, as the transgenic plant produced by the method according to the invention does not exhibit hypersensitive expression of PR genes when wounded and/or challenged by a pathogen and thus does not get necrotic lesions. The term “hypersensitive expression” or “hypersensitive response” relates to an induced response by which the plant deprives the pathogen of living host cells by localised cell death at the site of attempted pathogen ingress.

By way of another illustration, an agronomically important plant may in accordance to the method of the invention be stably transformed with the above mentioned antisense or mutant MAPK4 transcribed regions and their expression induced for the purpose to control or regulate plant growth, e.g. in cereal crops or in ornamental plants. In addition, the same MAPK4 regions could be placed under the control of a developmentally regulated or tissue-specific promoter in order to decrease the growth of the plant at a given developmental stage or to decrease the growth of a specific organ or tissue. By way of another illustration, dwarf plants could be produced for the purpose of bonsai culture by selecting for homozygous MAPK4 null alleles or by constitutive expression of an MAPK4 antisense. The terms “control” or “regulate” are used interchangeably with respect to size and relate to increasing or decreasing of the vegetative size of the plant.

The MAPK4 gene may be placed under a powerful constitutive promoter in the DNA construct. In useful embodiments of the method according to the invention the promoter is a constitutive promoter selected from the group consisting of cauliflower mosaic virus 35S promoter, cauliflower mosaic virus 90 with G-box 10 tetramer promoter (Ishige-Fumiharu et al. 1999), maize Adh promoter (Last et al., 1991), maize ubiquitin Ubi-I promoter (Christensen & Quail, 1996) and rice Act1 promoter (McElroy et al., 1990). By the term “constitutive promoter” is meant a promoter that is active all the time and does not require any specific stimulus for its activation.

There is considerable commercial interest in identifying the molecular “switches” which respond to non-hazardous chemicals applied to the plant, and in turn regulate developmental and defence responses where and when they are applied. Thus, transgenic plants expressing the MAPK4 gene under an inducible promoter are also contemplated to be within the scope of the present invention. Thus, in preferred embodiments of the method according to the invention the promoter is an inducible promoter, e.g. selected from the group consisting of the tetracycline repressor/operator controlled promoter, ecdysone agonist inducible promoter (Martinez et al., 1999), glucocorticoid agonist inducible promoter (Aoyama & Chua, 1997), copper inducible promoter (Mett et al. 1993), ethanol inducible promoter (Caddick et al. 1998), and tobacco wun 1 promoter (Seibertz-B et al., 1989). The term “inducible promoter” relates to promoters which are activated in the presence of a specific agent (the inducer), which may be a chemical compound or a physical stimulus such as heat or light. The chemical compound may be a chemical regulator which is not normally found in the plant in an amount sufficient to effect activation of the promoter, and thus the transcription of the DNA construct, to the desired degree at the time desired.

The use of the MPK4 gene described herein to produce agronomically important plants may have particular value as mentioned above. By way of illustration, and not by limitation, an agronomically important plant may be stably transformed with a chimeric gene containing e.g. one of the above inducible promoters driving the expression of the whole or a part of the transcribed region of an MAPK4 in the anti-sense orientation. If the inducer is applied to the plant during a period of pathogen attack, induced MAPK4 antisense expression will reduce expression or accumulation of endogenous sense MAPK4 mRNA, thereby reducing levels of endogenous MAPK4 protein. As MAPK4 represses the expression of downstream PR genes, chemical induction of the antisense will lead to expression of PR genes and hence increased pathogen protection.

In a further example, an agronomically important plant may be stably transformed with a chimeric gene containing an inducible promoter driving the expression of a catalytically inactive MAPK4 mutant protein. If the inducer is applied to the transgenic plant, inactive MAPK4 will compete with endogenous, activatable MAPK4, thereby leading to expression of downstream genes. This mechanism is explained in detail below.

The methods according to the invention are useful in enhancing resistance to disease-causing pathogens in both monocotyledonous plants (“monocots”) or dicotyledonous plants (“dicots”). Examples of commercially agronomically important plants wherein the method according to the invention may be useful include, but are not limited to, monocots such as rice, wheat, barley, rye, corn, maize or asparagus, and dicots such as avocado, apple, apricot, banana, bean, blackberry, broccoli, cabbage, carrots, cauliflower, celery, cherry, chicory, cucumber, garlic, grape, lettuce, mango, melon, nectarine, onion, papaya, parsley, pea, peach, pear, pepper, pineapple, plum, pumpkin, potato, radish, raspberry, squash, spinach, strawberry, soybean, sweet potato, tobacco, turnip, zucchini and pot plants and ornamental plants such as e.g. pelargonium, petunia, geranium, roses, tulips, daffodil, pink or lily. It will be understood that useful plants further include trees such as e.g. bonsai trees and dogwood.

As mentioned above transgenic plants produced by the method according to the invention and comprising the inactive MAPK4 sense transcripts may also exhibit visible, phenotypic abnormalities including reduced size. Thus, in one useful embodiment of this aspect of the invention the transgenic plant has, relative to the wild type plant, reduced growth.

As mentioned above, although the use of the Arabidopsis thaliana MAPK4 cDNA (AtMPK4) is exemplified herein, this invention is intended to encompass the use of MAPK4 nucleic acids and MAPK4 proteins, as well as corresponding antisense nucleic acid sequences and nucleic acid sequences encoding catalytically inactive MAPK4 proteins, from other plant species that are sufficiently similar to be used instead of the Arabidopsis MAPK4 for the purposes described herein. This includes the use of antisense nucleic acid sequences and nucleic acid sequences encoding catalytically inactive MAPK4 proteins derived from MAPK4 nucleic acid molecules that encode the MAPK4 polypeptide having at least 70%, preferably at least 80% and most preferably at least 90% homology with the Arabidopsis MAPK4 deduced amino acid disclosed in Mizoguchi et al. (1993), the homology being as defined above.

In a further aspect the present invention relates to a transgenic plant having enhanced wound and/or disease resistance, said plant comprising an antisense MAPK4 construct, wherein said construct leads to an increase in the expression of wounding and/or pathogen responsive genes.

In another aspect the invention relates to a transgenic plant having enhanced wound and/or disease resistance, said plant comprising a constitutively active form of MAPK4.

In a still further aspect the invention relates to a transgenic plant having enhanced wound and/or disease resistance, said plant comprising a catalytically inactive MAPK4 construct, wherein said construct leads to an increase in the expression of wounding and/or pathogen responsive genes. In a further aspect, the invention provides a transgenic plant having enhanced wound and/or disease resistance, said plant comprising a mutation in the MAPK4 gene which results in a loss of function of said gene, wherein said mutation leads to an increase in the expression of wounding and/or pathogen responsive genes. The transgenic plants according to the invention have, relative to the wild type plants, reduced growth. In preferred embodiments the transgenic plants according to the invention are monocots or a dicots.

In a further aspect the present invention relates to a recombinant DNA construct comprising the coding region of MAPK4 gene operably linked in an antisense orientation to an appropriate promoter. It will be appreciated that a useful promoter may be selected from the constitutive or inducible promoter described above.

In a still further aspect the invention relates to a transgenic plant cell transformed with the DNA construct according to the invention.

It is another aspect of the invention to provide a method for screening a plant population for plants carrying an insertion element within the MAPK4 gene whereby the gene is functionally inactivated, the method comprising the steps of a) providing a MAPK4 specific primer and an insertion element specific primer, b) providing DNA of each of said plants, c) performing PCR reactions using said primers and a templates DNA of each of said plants, and d) selecting a plant carrying an insertion element within the MAPK4 gene whereby the gene is functionally inactivated by identifying a PCR product primed by said primers. Such methods for screening a plant population are generally recognised in the art as a “reverse genetic screen or analysis” and/or as a “gene machine”.

It will be understood, that by providing the sequence of the MAPK4 for a specific plant species and by providing the sequence of a useful insertion element, it is possible to find within said specific plant population a plant which have an enhanced wounding and/or pathogen resistance and/or have a reduced growth relative to the wild type plant.

Furthermore, the invention pertains to the use of a MAPK4 gene for providing MAPK4 primers useful in the method according to the invention.

The invention will now be described in further details in the following non-limiting example and the drawings wherein

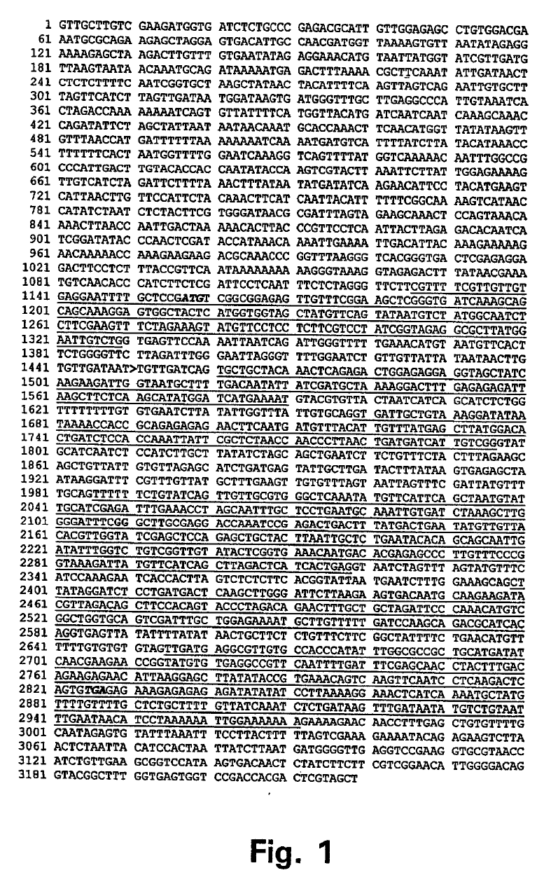

FIG. 1 shows the MPK4 genomic fragment amplified by primers (18mers at ends of this sequence: no name BamH1 linker and p3y). The sequence is the complement of nt4700-7918 of BAC IG002N01 (GENBANK/EMBL accession AF007269, NID 2191126 deposited Dec. 6, 1997. According to accession, gene starts at 130 of this sequence. Underlined are exons from cDNA (accession D21840, NID 457399), bold are start, stop, and > is Ds insertion site;

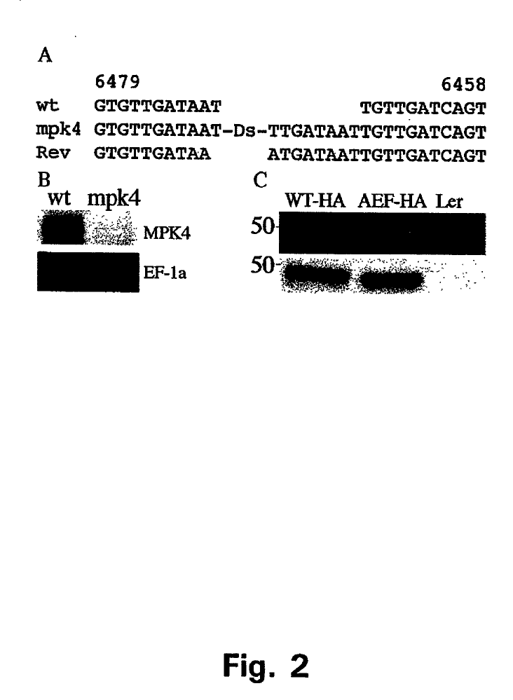

FIG. 2 shows in (B) the sequence of the MPK4 first intron with acceptor site (AGT) from wild type Ler. The numbers above are base pairs from the same sequence of wild type Col-0 (complement of GI:2191126; ABB61033). Middle, the 8 bp Ds target site insertion in mpk4 (bold). Bottom, the 7 bp footprint with a single nucleotide change in the revertant produced by Ds excision. (C) Northern blot of 10 μg total RNA from wild type (wt) and mpk4 probed with radio-labelled MPK4 cDNA and EF-1 cDNA as a loading control. (D) Top, kinase activities immuno-precipitated from mpk4 expressing wild type HA-tagged MPK4 (WT-HA) and mutated MPK4 (T201A/Y203F; AEF-HA). Ler control is wild type without HA tagged MPK4. Bottom, western blot of the same immuno-precipitates using anti-HA antibodies;

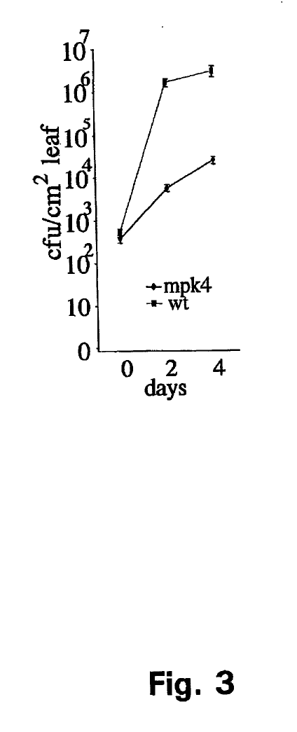

FIG. 3 shows the resistance of mpk4 to bacterial and oomycete pathogens. (A) Four-week-old wild type and mpk4 plants were inoculated with the virulent strain DC3000 of Pseudomonas syringae pv. tomato at a concentration of 1×10⁵ colony forming units per ml (cfu/ml). Values represent average and standard deviations of cfu extracted from leaf disks in three independent samplings;

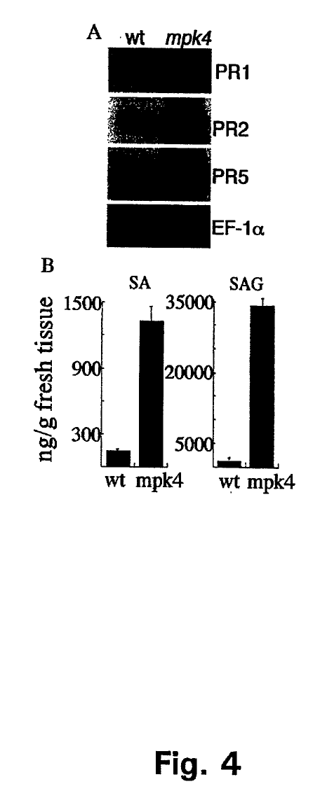

FIG. 4 shows the accumulation of PR mRNAs and SA in wild type and mpk4. (A) RNA gel blots of 10 μg total RNA from wild type (wt) and mpk4 probed with radiolabelled PR1, PR2, PR5, and EF-1α loading control. (B) Leaves from 4-week-old plants grown in soil were harvested and free SA and SAG contents (ng/g fresh weight) quantified by HPLC;

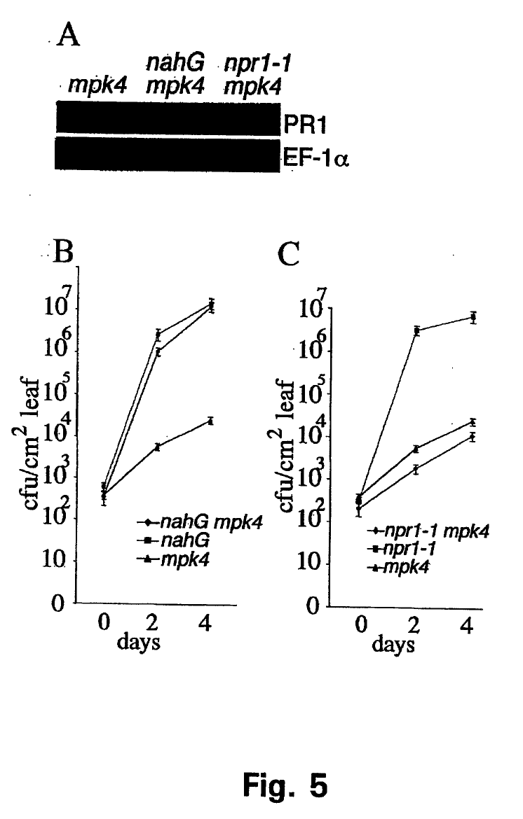

FIG. 5 shows in (B) a RNA gel blot showing the accumulation of PR1 mRNA in mpk4, homozygous mpk4 expressing NahG, and the npr1-1/mpk4 double mutant. EF-1α (bottom) is the loading control. (C) Growth of the virulent strain DC3000 of Pseudomonas syringae pv. tomato after inoculation into nahG/mpk4 and the parental nahG and mpk4 lines. Experimental conditions were as described in FIG. 3A. (D) Similar experiment as (C) carried out on the npr1-1/mpk4 double mutant and parental lines;

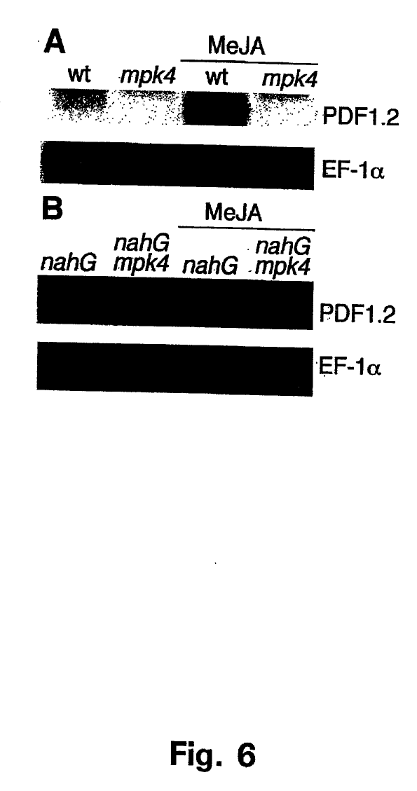

FIG. 6 shows the accumulation of PDF1.2 mRNA in wild type, mpk4 mutant and in plants expressing nahG. (A) Northern blot showing the accumulation of the JA-inducible PDF1.2 mRNA in wild type (wt) and mpk4. EF-1α (bottom) is the loading control. (B) Accumulation of PDF1.2 mRNA in wild type and mpk4 mutants expressing nahG.

### EXAMPLE 1

Generation of the G16 Transposant Line and Identification of the Recessive Insertion Mutation

1.1 Introduction

The identification of mutant phenotypes caused by gene knockouts is an important first step in elucidating the functions of corresponding genes (Miklos & Rubin, 1996). As targeted gene knockout by homologous recombination is not currently practicable in plants, phenotypes caused by knockouts are identified by genetic or reverse genetic approaches. In both approaches, transposable elements (Sundaresan et al. 1995) and T-DNAs (Feldman et al. 1989) are used as insertional mutagens because their sequences can be used as tags to isolate and identify the insertion site, and hence the gene disruption, by standard cloning techniques or current PCR-based methods (Liu et al. 1995). In genetic approaches, these elements are randomly introduced into the genome and mutants scored phenotypically by visual examination or by more specific physiological or biochemical assays. For example, an engineered maize Ds transposon is widely used for insertional mutagenesis in the model plant Arabidopsis (Sundaresan et al. 1995). This system is initially time consuming due to the low transposition frequency of Ds in Arabidopsis, and because it entails phenotypic screening in stable F3 transposant lines.

Other modified transposons and T-DNAs can be used to generate larger numbers of insertions, which makes them useful in reverse genetic screens (Krysan et al. 1996). In this strategy, insertions in a specific gene of interest can be identified by screening individual or pooled plant DNA by PCR with gene-specific and T-DNA- or transposon-specific primers. A drawback of the high frequency insertion systems is that backcrossing is generally required to obtain lines carrying single site insertions for mutants analysis. For both transposon and T-DNA insertions, specific, loss of function gene disruption can be assigned to the phenotype by complementation of the mutant with the wild-type gene. In addition, this assignment can be made for transposon mutants by obtaining wild-type revertants after excision of the transposon.

Here we describe a recessive Arabidopsis mutant, caused by the insertion knockout of the MAP kinase 4 gene (Mpk4) (Mitzoguchi et al. 1993), which was identified by visual examination of stable transposant lines generated with a modified maize Ds transposon. This mutant exhibits constitutive defence responses without spontaneous necrotic lesions, including elevated SA levels and resistance to oomycete and bacterial pathogens. RNA blot and cDNA microarray hybridizations demonstrate that the mutant constitutively expresses PR genes normally induced by SA and fails to induce PDF1.2 and THI2.1 mRNA in response to JA. Molecular cloning, revertant analysis and complementation studies demonstrate that the phenotype of the mutant (mpk4) is caused by loss of MPK4 activity. These data suggest a role of MPK4 in regulating plant defenses against pathogens.

1.2 Materials and Methods

1.2.1 Isolation and Characterization of mpk4

Transposant lines were generated as described by Sundaresan et al. (1995) using the transgenic parental lines in the Ler ecotype obtained from the Arabidopsis Information Service (http://aims.cps.msu.edu/aims/) expressing maize Ac transposase (Ac1, order #CS8043/N8043) and carrying an engineered maize Ds gene trap cassette (DSG1, order # CS8046/N8046). F3 seeds from individual selfed, F2 plants (named G1-n for gene-trap in the order F2 plants were harvested) were selected on kanamycin. A minimum of 12 of these plants were then transferred to soil and allowed to grow to maturity in a green house. Plants were examined visually during growth and flowering for phenotypic abnormalities.

For scanning electron microscopy, leaves were fixed overnight at 5° C. in 0.1 M phosphate buffered (pH 7.0) 2.5% glutaraldehyde and 2% paraformaldehyde, postfixed in 1% OsO₄, dehydrated in acetone, critical-point-dried via CO₂, gold sputter-coated, and examined in a Philips 515 scanning electron microscope.

1.2.2 Analysis of the mpk4 Mutant and MPK4 Alleles

DNA manipulations were performed after standard procedures (Maniatis et al., 1982). More specialised protocols relating to plant growth, nucleic acid extraction and purification and plant transformation are described by the inventors electronically at http://genome-www.stanford.edu/Arabidopsis/Protocols Mundy2.html. The transposon Ds insertion region was identified by Southern blotting of genomic DNA from the G16 dwarf progeny digested with EcoRI (single site in Ds) probed with the E. coli UidA (GUS) gene carried on the end of Ds (Sundaresan et al., 1995). A 4.5 kb hybridising fragment (SEQ ID NO:1) including 2.4 kb GUS and Ds sequences and 2.1 kb flanking genomic sequence was purified by gel electrophoresis, ligated to EcoRI digested lambda gt11 arms, and the flanking region between Ds and the vector arm amplified with a long range PCR kit (Perkin-Elmer) using a vector primer (5′CAGACCAACTGGTAATGGTAGCG, SEQ ID NO:3) and a GUS primer (5′ CTGCATCGGCGAACTGATCG, SEQ ID NO:4) (FIG. 1). Sequencing of both strands on an Applied Biosystems ABI 310 and comparison of these sequences using BLAST (Altschul et al. 1990) at the National Center for Biotechnology Information (http:/www.ncbi.nlm.nih.gov/BLAST) revealed that Ds was integrated 8 nt upstream of the acceptor site of the first intron of the AtMpk4 gene (nucleotide 6489, gene encoded on complement of GENBANK/EMBL accession GI: 2191126, corresponding to the AtMPK4 cDNA described by Mizoguchi et al., 1993). Genomic DNA from wild type and mpk4 revertants was used as template to amplify the region spanning the putative footprint, and these fragments sequenced as above.

1.2.3 Pathogen Infection, Salicylic Acid (SA) Measurement and Methyl Iasmonate (MeJA) Treatments

Four-week-old plants were infiltrated with a suspension of 1×10⁵ cfu/ml of virulent Pseudomonas syringae pv. tomato DC 3000 strain and bacterial growth assayed (Parker et al., 1996). Inoculations with P. parasitica isolate Cala2 were performed on seedlings, and monitored by staining with lactophenol-tryphan blue (Parker et al., 1997). SA and SA glucoside were measured in leaves of 4 week-old plants (Bowling et al., 1994). For MeJA treatments, 4-week-old greenhouse plants were either sprayed with 50 μM MeJA solution, containing 0.005% Silwet L-77 or water and 0.005% Silwet L-77 and harvested after 48 hours.

1.2.4 RNA Analyses

Total RNA was prepared for RNA gel blot hybridisation using standard protocols (RNA-gents®Total RNA, Promega). Probe templates were amplified by PCR from cDNAs or genomic DNA with primer sequences from: MPK4 (GI:457399), PR1 (GI:4454853), β-1,3-glucanase or PR2 (GI:166637), PR5 (GI:6646759), PDF1.2 (GI: 4759674), THI2.1 (GI: 1181530) and elongation factor 1α control (GI:16260). For cDNA microarray analysis, total RNA from 2 g of 18 day old, soil grown wild type and mpk4 was extracted using Trizol Reagent (Life Technologies). Poly(A)⁺ RNA was purified from 200 μg total RNA with 2 μg of Dynabeads Oligo(dT)₂₅ (Dynal). cDNA microarray production, preparation of fluorescent probes and microarray hybridisation and scanning have been described previously (Ruan et al., 1998). The hybridisation experiment was performed twice using microarrays hybridised to cDNAs from two samples each of mpk4 and wild type mRNA.

1.2.5 Microarray Data Analyses

Analysis of microarray hybridisation signals using the average log₂ fold balanced difference between mpk4 and wild type signals, and its standard deviation, indicated that clones with ≧5-fold signals in mpk4 than wild type were statistically significantly higher expressed in mpk4 (P<0.01). 5′ upstream sequences of PR1, PR5 and of the cDNAs shown by the microarray to be ≧5-fold higher expressed in mpk4 than wild type were extracted from the database (Table I). One of these (unknown) was not included because we could not determine the ORF start. The 17 putative promoter regions were used as input in a Gibb's sampler, which can detect short patterns or matrices that are not necessarily 100% conserved (Lawrence et al., 1993), to identify sequences which might be regulatory cis-elements. Searches were performed for elements ranging from 6 to 16 bp. The sampler repeatedly found Matrix 1 (TTGACT) and Matrix 2 (GACTWWHC) when searching for elements of 6 or 8 bp, respectively. The best matrices found for 7, 9, 10, 11 and 12 bp all had similarity to Matrix 2, but lower information content.

To estimate the statistical significance of these matrices or motifs, the nucleotides in the 17 promoter sequences were shuffled 300 times, producing 300 sets of 17 promoter sequences of conserved lengths and nucleotide compositions. Gibbs sampling was performed on each of the shuffled sequence sets for both 6 and 8 bp elements, and the total information content for the best matrix was collected. The information content for the 300 best matrices was approximately Gaussian distributed, with a mean of 7.7 bit and 8.8 bit and a standard deviation of 0.27 bit and 0.25 bit, and with the highest information content found 8.5 bit (twice) and 9.6 bit (once) for the 6 and 8 bp matrices respectively. The total information contents of 8.6 bit for Matrix 1 and 9.7 bit Matrix 2 were therefore significantly (P<0.01) higher than expected by random. Thus, there is by random less than 1% chance of finding any 6 bp or 8 bp motifs as conserved as Matrix 1 & 2 in sets of DNA sequences of the same length and nucleotide composition as the 17 promoters. Detailed protocols and microarray results are available upon request from the authors.

1.2.6 Genetic Analyses

F1 progeny of crosses between mpk4 and npr1-1 plants were selfed, and F3 seeds from 60 individual F2 plants plated on MS with 250 μM SA. This allowed the identification of 26 npr1 homozygotes by seedling hypersensitivity to SA (Bowling et al., 1997). Lines homozygous for npr1 were tested for Ds in mpk4 by PCR. 40 F3 seeds from plants homozygous for npr1 and heterozygous for mpk4 were grown in soil. All of these lines segregated for the mpk4 dwarf phenotype indicating that mpk4 dwarfism was independent of npr1. All homozygous npr1 dwarves examined expressed PR1 constitutively and were confirmed by sequencing as homozygous for npr1-1.

Plants homozygous for the nahG salicylate hydroxylase were crossed to mpk4 heterozygotes, and F1 progeny heterozygous for Ds in mpk4 identified by PCR. 300 F2 progeny were examined for mpk4 homozygous dwarfism. Since the nahG hydroxylase is dominant, 75 dwarves would be expected if the activity of NahG did not suppress the mpk4 phenotype. In contrast, 19 dwarves would be expected if nahG rescued mpk4 dwarfism. Only 22 dwarves were identified, while 48 plants had a partially suppressed mpk4 phenotype. PCR demonstrated that the nahG transgene was absent in all of the 22 dwarves, whereas plants exhibiting partially suppressed dwarfism were homozygous for mpk4 and carried nahG.

1.2.7 HA-Tagging, Immuno-Detection and in Gel Kinase Assay

A NotI linkered genomic MPK4 fragment including the 1150 bp promoter was amplified from La-0 genomic DNA and cloned into that site of pSLF172 (Forsburg and Sherman, 1997) to produce a C-terminally triple HA-tagged MPK4. The activation loop mutant (T201A/Y203F) was made using the QuickChange kit (Stratagene). HA-tagged mutant and wild type MPK4 were subcloned into pCAMBIA3300, and transformed into mpk4 heterozygotes. Homozygous mpk4 lines expressing HA-tagged MPK4 versions were identified in T2.

Protein extracts were prepared as described (Romeis et al., 1999), except that no buffer change was made prior to immunoprecipitation. 100 μg of total protein was immunoprecipitated with 2 μg/ml monoclonal 12CA5 HA-antibody (Boehringer) as described (Romeis et al., 1999). In-gel kinase assays were performed as previously described (Zhang and Klessig, 1998). Western blots were developed using alkaline phosphatase conjugated anti-mouse antibody (Promega).

1.2.8 MPK4 Localization

An 1150 bp 5′ upstream fragment containing the MPK4 promoter was isolated as a BamH1/HindIII-linkered PCR product and transcriptionally fused upstream of GUS in pCAMBIA3300. Plants were transformed by vacuum infiltration, and transgenics selected with BASTA.

RT-ISPCR on FAA fixed leaves was performed according to Johansen (1997) without pepsin and DNase treatment. MPK4 mRNA-specific primers spanning exons I were used for reverse transcription and PCR amplification. An anti-DIG-AP Fab fragment (Boehringer Mannhheim) was used for detection.

1.3 Results

1.3.1 Analysis of the mpk4 Mutant and MPK4 Alleles

The mpk4 mutant is a dwarf identified among stable transposant lines generated with a modified maize Ds element (Sundaresan et al., 1995). mpk4 has curled leaves, and flowers with reduced pollen production and fertility. Due to the infertility of the dwarf plants, the mutation was maintained in the heterozygous state during subsequent generations. Microscopy revealed that mpk4 dwarfism was caused by decreased cell size. mpk4 seed germinated with normal cotyledons and first exhibited dwarfism at the two to three leaf stage. Dwarf leaves had normal numbers of cells of significantly reduced size (≅20-30% of heterozygote or parental wild type) and normal numbers of stomates, many with a distinct, donut shape. Reduced epidermal cell size of the dwarf resulted in increased numbers of stomates per surface area (≅1.8 times wt). Perfusion chamber tests indicated that stomates of the dwarf did not close fully in response to water-stress (≅28% of wt). This effect caused the dwarf to wilt faster than wild type when dehumidified. No necrotic lesions were detectable on mpk4. In progeny of mpk4 heterozygotes, the recessive dwarfing allele co-segregated with Ds-encoded kanamycin resistance. To identify this allele, genomic DNA flanking Ds was isolated and sequencing revealed that Ds was integrated 8 nt upstream of the acceptor site of the first intron of MPK4 (FIG. 2A).

Three approaches demonstrated that this insertion was responsible for the mpk4 phenotype.

First, revertants were generated by Ds excision following crosses to a line expressing Ac transposase. This identified wild type F3 plants homozygous for kanamycin resistance.

Genomic fragments were amplified from revertants, wild type, and mpk4. Sequencing revealed that Ds had created an 8 bp target site duplication on insertion in the MPK4 intron, and that a 7 bp footprint remained after Ds excision to restore the transcription unit (FIG. 2A, SEQ ID NO: 5-10). Thus, transposition away from MPK4 is linked to reversion of the dwarf phenotype. Second, RNA blot hybridization showed that mpk4 homozygotes did not accumulate detectable MPK4 mRNA, in contrast to wild type (FIG. 2B) as well as the revertant (not shown). Third, mpk4 mutants were rescued by transformation with a 3.3 kbp fragment containing MPK4 and 1150 bp of 5′ upstream and 506 bp of 3′ downstream sequence. In addition, mpk4 was complemented with the same genomic fragment containing a triple HA-epitope tag at the C-terminus of MPK4. Western blotting and in gel kinase assay showed that MPK4 is active in wild type plants (FIG. 2C). In contrast, equivalent levels of a catalytically inactive, HA-tagged MPK4 containing two mutations in activation loop residues (T201A/Y203F) had no effect on the mpk4 phenotype (AEF-HA; FIG. 2C). These results demonstrate that the mpk4 phenotype is caused by loss of MPK4 kinase activity.

1.3.2 Analysis of mpk4 Responds to Growth Regulators and Abiotic Stresses

Experiments were performed to determine a function or functions of MPK4. Growth response assays and northern hybridisation with target genes indicated that mpk4 was not significantly impaired in responses to environmental stresses including desiccation, salt-treatment, cold or heat shock. mpk4 responses to phytohormones controlling germination and growth including auxin (Gray et al., 1998), cytokinin (Vogel et al., 1998), brassinosteroid (Li & Chory, 1997), abscisic (Leung et al. 1994), jasmonic (Xie et al., 1998), and gibberellic (Peng et al., 1997) acids. This suggests that the mpk4 phenotype is not caused by defects in responses to any of these abiotic stresses and phytohormones. However, mpk4 leaf size and stomatal shape were similar to that of ethylene constitutive response (CTR) mutants (Hua & Meyerowitz, 1998). Nonetheless, mpk4 did not exhibit a full seedling CTR, and mpk4/ctrl-1 double mutants exhibited extreme dwarfism than either mutant parent. Similarly, mpk4/ein2-2 double mutants exhibited mpk4 dwarfism and ein2-2 insensitivity to ethylene (Alonso et al., 1999). This indicated that MPK4 does not act in the ethylene response pathway between CTR1 and EIN2.

The possibility that MPK4 is involved in stress signalling was also initially tested by comparing the accumulation patterns of stress responsive mRNAs in the mpk4 mutant versus wild type. This showed that mpk4 expression of the hyperosmotic responsive rab18 gene (Mantyla, et al., 1995) was normal, but that the mutant hyperaccumulated the HSP18.2 target gene in response to heat shock (Takahashi & Komeda, 1989), and hypoaccumulated the COR15a target gene in response to cold stress (Artus et al., 1996). This indicates that mpk4 is compromised in certain responses to temperature stress.

1.3.3 mpk4 Exhibits Increased Resistance to Pathogens

Constitutive defense response mutants such as cpr1, ssi1, and lsd6 exhibit dwarfism and leaf curling similar to mpk4 (Bowling et al., 1994; Shah et al., 1999; Weymann et al., 1995), so the resistance of mpk4 to pathogens was examined. It was found that mpk4 is highly resistant to a virulent bacterial pathogen, Pseudomonas syringae pv. tomato DC3000, and to infection by a virulent isolate of the oomycete pathogen, Peronospora parasitica (Cala2; Parker et al., 1996). This pathogen rapidly colonised and caused disease symptoms on wild type plants but was undetectable in mpk4 plants (FIG. 3A). Thus, mpk4 exhibits enhanced resistance to at least two unrelated types of pathogens.

1.3.4 mpk4 Expresses PR Genes Constitutively

Since mpk4 exhibited resistance to pathogens, the expression of PR genes in mpk4 and wild type was compared. RNA blots demonstrated that PR1, PR2 and PR5, that are normally induced during the development of SAR (Glazebrook, 1999), were constitutively expressed in mpk4. This suggests that MPK4 negatively regulates the expression of these PR genes. In addition, mpk4 expressing the inactive T201A/Y203F MPK4 form expressed PR1 to the same level as the knockout mutant (data not shown), indicating that MPK4 activity is required for the negative regulation of PR gene expression.

In order to identify a more complete set of downstream genes in MPK4 signalling, global gene expression in mpk4 and wild type seedlings by cDNA hybridisation to a microarray of 9,861 cDNAs expressed throughout Arabidopsis development was compared (Ruan et al., 1998). This revealed that, of the 7864 (80%) seedling cDNAs which hybridised, the majority hybridised at roughly equivalent levels in mpk4 and wild type (7707 or 98% between 3-fold over- and underexpressed). Only 16 cDNAs (=0.2%) exhibited greater than five-fold differences in hybridisation levels between mpk4 and wild type (Table 1). All 16 were more highly expressed in mpk4 suggesting that MPK4 is involved in the repression of a subset of genes. Database analysis showed that while the function of eight of these remain to be elucidated, eight encode well known PR or wound-induced proteins (Glazebrook et al., 1997). These include chitinase and β-1,3-glucanases (PR2) which have antifungal activities, extensin and pectin methylesterase involved in cell wall modification (Merkouropoulos et al., 1999), and glutathione-S-transferases, ascorbate reductase (Grantz et al., 1995), and oxalate oxidase (Zang et al., 1995), the latter potentially involved in oxidative cell wall cross-linking. In addition, lipid transfer proteins may contribute to plant defense (Molina and Olmedo, 1997), and LRR receptor kinases are involved in plant pathogen signalling (Glazebrook et al., 1997).

It has been reported previously that elicitor treatment of tobacco rapidly induces local and systemic expression of endoplasmic reticulum (ER)-resident folding chaperones including luminal binding protein (BiP), protein disulfide isomerase (PDI) and calreticulin (CRT; Jelitto-Van Dooren et al., 1999). This may prepare the ER for the massive upregulation of secreted PR-proteins. The microarray analysis showed that mpk4 also exhibits this response. cDNAs encoding homologues of the heat shock proteins HSP70 and HSP90 and of the reticuloplasmins BiP, PDI, CRT and calnexin, were between 3.5 and 4.5 more highly expressed in mpk4 than in wild type (not shown).

The constitutive expression of the PR genes suggests that a pathway in which MPK4 participates may regulate the activity of a transcription factor or complex controlling PR gene expression. 5′ upstream sequences of 17 of these genes (15 from the microarray and PR1 and PR5) could be identified in the database. These sequences were searched for the occurrence of conserved sequence motifs which might be binding sites for common regulatory factors. Two consensus sequences were identified with statistically significant frequencies of occurrence (Table 1). One of these sequences, TTGACT (P<0.01), is a negative regulatory element in the Arabidopsis PR1 promoter (LS4; Lebel et al., 1998), and a similar element binds an elicitor induced, WRKY transcription factor in the parsley PR1 gene (W-box; Eulgem et al., 1999). The other sequence (GACTWWHC, P<0.01; W=A/T, H=A/T/C) is similar to a positive regulatory element in the PR1 promoter (LS10 or GGACTTTTC; Lebel et al., 1998). In contrast, the third cis-element identified in the PR1 promoter (LS7 or G-box; Lebel et al., 1998; Zhang et al., 1999) did not occur at a statistically significant frequency in these putative promoters.

TABLE 1.1
mRNAs over-expressed in mpk4 seedlings

| | Accession | Gene or Homologue |
|---|---|---|
| Fold mpk4/wt | GI number | Description |
| Northern blot | 4454853 | PR1 |
| | 6646759 | PR5 |
| Microarray | | |
| 29.3 | 2288989 | Chitinase |
| 23.8 | 1167961 | Extensin (EXT1) |
| 19.2 | 166637 | β1,3-glucanase (BGL2/PR2) |
| 11.6 | 553038 | β1,3-glucanase (BGL3) |
| 11.3 | 1890156 | Glutathione S-transferase (ERD11) |
| 8.4 | 3461818 | Glutathione S-transferase (PM24) |
| 7.5 | 6143882 | Monodehydroascorbate reductase |
| 7.4 | 2160155 | Unknown |
| 6.9 | 903895 | Pectin methylesterase (PME1) |
| 6.4 | 9758178 | Lipid transfer protein (MTE17.7) |
| 6.2 | 4887748 | LRR-receptor kinase |
| 6.2 | 2462835 | Hypothetical protein |
| 5.4 | 7267528 | LRR-receptor kinase |
| 5.3 | 3482931 | Oxalate oxidase-like (GLP5) |
| 5.3 | 7269612 | Stomatin-like |
| 5.3 | 6686401 | Proline-rich, hypothetical protein |

1.3.5 SA-Dependent Signaling in mpk4

Since SA is necessary and sufficient for SAR, levels of SA and SA glucosides (SAG) were compared in wild type and mpk4. This showed that SA and SAG levels were 9- and 25-fold higher in mpk4 (FIG. 4B). The SA and SAG levels in mpk4 are similar to those in cpr1 (Bowling et al., 1994). Although both CPR1 and MPK4 are on chromosome 4, progeny analysis of crosses between cpr1 and mpk4 mutants demonstrated that they are not allelic (data not shown).

Genetic approaches were used to determine whether the mpk4 phenotype was SA-dependent. Double heterozygous F1 progeny of crosses between homozygous nahG plants and plants heterozygous for Ds were identified by PCR. All dwarf F2 progeny were then shown to lack nahG by PCR. In addition, plants were identified with a partial suppression of mpk4 dwarfism. All such plants were homozygous for mpk4 and contained the nahG transgene. The ability of nahG to suppress the mpk4 phenotype was confirmed by measuring resistance towards Pseudomonas syringae pv. tomato DC3000 and the level of PR1 expression in mpk4/nahG plants. This revealed that both bacterial resistance and PR1 gene expression in mpk4 were fully dependent upon SA (FIGS. 5A&B). Therefore, MPK4 functions upstream of SA in SAR signaling.

The npr1-1 mutant is blocked in SA-mediated induction of PR genes (Cao et al., 1994). To examine whether npr1-1 is epistatic to mpk4, the phenotypes of mpk4/npr1-1 double mutants were examined. The double mutant fully retained mpk4 dwarf stature, constitutively expressed PR1, and exhibited bacterial resistance as mpk4 (FIGS. 5A&C). In addition, it exhibited the SA hypersensitivity typical of npr1-1 seedlings (Bowling et al., 1997). Thus, either MPK4 and NPR1 participate in two different pathways leading to SAR, or MPK4 functions downstream of NPR1.

1.3.6 Gene Induction by Jasmonate is Blocked in mpk4

PR gene overexpression in mpk4 was the most striking difference revealed by the microarray analysis. However, eight genes hybridised >3 times less intensely to mpk4 than wild type cDNA. The most affected of these (Acc#4587541; <3.7 fold wild type) encodes a homologue of a myrosinase associated protein from Brassica napus (MyAP; Taipalensuu et al., 1997). MyAP expression is induced by wounding and JA but is repressed by SA. JA is an important secondary signal in plant defense responses, and there is evidence for specific crosstalk between SA and the JA and ethylene signaling pathways (Pieterse and van Loon, 1999). Therefore, the expression of PDF1.2 and THI2.1, two JA-response genes, in wild type and mpk4 was compared. It was found that mpk4 does not express PDF1.2 or THI2.1 constitutively, unlike the cpr mutants cpr5, cpr6 and ssi1 (Bowling et al., 1997; Clarke et al., 1998; Shah et al., 1999). More significantly, methyl jasmonate (MeJA) treatment failed to induce the expression in mpk4 of PDF1.2 (FIG. 6A) and THI2.1 (data not shown). Since this could result from high SA levels antagonizing JA signalling (Felton et al., 1999; Gupta et al., 2000), PDF1.2 and THI2.1 mRNA accumulation after MeJA treatment was examined in wild type and mpk4 expressing nahG. This revealed that while PDF1.2 and THI2.1 mRNAs were induced by MeJA in nahG expressing wild type, they were not inducible in mpk4/nahG (FIG. 6B; THI2.1 not shown). These results indicate that MPK4 is required for PDF1.2 and THI2.1 expression in response to MeJA, irrespective of the levels of SA in the plant.

1.3.7 Expression and Localization of MPK4

The expression pattern of MPK4 was examined in transgenic plants carrying a transcriptional fusion between the GUS reporter and the same 1150 bp of 5′ upstream MPK4 sequence used to drive the expression of the complementing genomic clones. In soil-grown plants, strong GUS activity was detected in the veins and stomatal guard cells of leaf plates, petioles, stem and flowers, while leaf mesophyl cells showed weaker staining. The leaf expression pattern was confirmed by in situ PCR with MPK4 cDNA specific primers which detected highest levels of MPK4 mRNA in phloem, leaf edges and stomata. No signal was detected in non-reverse transcribed wild type leaves, or in leaves of mpk4 (data not shown), confirming the specificity of the reaction. Vein and guard cell expression is shared by the SAR target gene PR2 in a cpr1 background (Bowling et al., 1994), and vein expression by the putative SAR regulator, SNI1 (suppressor of no-immunity; Li et al., 1999). Curiously, in sterile-grown seedlings, GUS expression was largely confined to stomatal guard cells.

1.4 Discussion and Conclusion

Interactions between plants and pathogens involve recognition and signaling events that are distinct for different pathogen elicitors. However, many of these initial signals are integrated into convergent defense pathways (Yang et al., 1997; Glazebrook, 1999). One such pathway leads to the development of SAR, for which SA is a necessary and sufficient host signal.

Evidence reported here shows that the mpk4 mutant exhibits constitutive SAR. Loss of MPK4 function leads to increased SA levels and, similar to other SA-accumulating mutants, mpk4 exhibits enhanced resistance to virulent pathogens. Furthermore, RNA blot analysis showed that mpk4 constitutively expresses molecular markers of SAR. This was confirmed by microarray analysis which showed that mRNAs corresponding to 16 of the 7684 (0.2%) displayed cDNAs expressed in seedlings were statistically significantly more highly expressed in mpk4 than in wild type. Eight of these 16 genes have been shown to be responsive to SA or induced by wounding and/or pathogen infection (Glazebrook et al., 1997). In addition, the 5′ upstream regions of these genes contain a consensus GACTWWHC motif and the W-box (TTGACT) involved in the control of Arabidopsis and parsley PR1 expression in response to elicitors and SA (Lebel et al., 1998; Eulgem et al., 1999). Thus, a specific set of effector genes involved in pathogen defense and SAR, presumably regulated via shared transcription factors, is constitutively expressed in mpk4.

Lesion-mimic mutants which constitutively express SAR and develop spontaneous necrotic lesions are common. Since coordinate activation of programmed cell death (PCD) and defense responses may result pleiotropically from disruption of cellular homeostasis (Mittler et al., 1995; Molina et al., 1999), the specific roles of genes defined by lesion-mimic mutations in defense signaling are uncertain. This raises the question of whether the constitutive SAR phenotype of mpk4 is a pleiotropic effect of disturbances of normal cell function in mpk4. However, several lines of evidence indicate that MPK4 specifically acts as a negative regulator of SAR. First, mpk4 does not exhibit necrotic lesions, and therefore does not fall into the common class of lesion-mimic mutants. The lack of spontaneous cell death in mpk4 is critical, since disruption of normal cell function might be expected to turn on PCD pathways. Second, if the mpk4 phenotype were a pleiotropic effect of an unbalanced biochemical state induced by the mpk4 mutation, a general activation of defenses, including SA and JA dependent pathways, might be expected. Simultaneous activation of SA- and JA-dependent defense pathways is seen in acd2, ssi1, cpr5 and cpr6 (Greenberg et al., 1994; Penninckx et al., 1996; Shah et al., 1999; Bowling et al., 1997; Clarke et al., 1998), as well as in tobacco plants expressing a bacterial proton pump (Mittler et al., 1995). However, in mpk4 these two major defense pathways are oppositely affected, since SA-dependent defenses are constitutively expressed, while induction of JA-dependent defense genes is blocked. Third, microarray hybridization showed no other obvious differences than in defense related transcripts, suggesting that SAR expression is the only deviation from homeostasis in mpk4. In addition, mpk4 responded normally to a range of abiotic stresses and phytohormones, and MPK4 is therefore not involved in responses to these stimuli. Fourth, MPK4 is constitutively active under normal conditions and its activity is required to repress SAR, since the inactive MPK4 mutant (T201A/Y203F) failed to complement the dwarf and PR1 expression phenotypes of mpk4. This argues that inappropriate crosstalk between MAPK isoforms in the mpk4 mutant is not the cause of constitutive SAR expression. Thus, SAR is negatively regulated by MPK4 kinase activity.

NahG abolishes PR gene expression and bacterial resistance in mpk4, and partially suppresses mpk4 dwarfism. This indicates that MPK4 functions upstream of SA in SAR signaling. Also, since dwarfism and constitutive expression of secreted PR-proteins appear to be linked, the basis for the dwarfism of mpk4 and other constitutive SAR mutants may include the metabolic cost of increased PR-protein synthesis and maintenance of a secretory pathway tuned for massive protein secretion. Incomplete suppression of dwarfism by NahG is also observed in the cpr1 and dnd1 mutants (Bowling et al., 1994; Clough et al., 2000), suggesting that other targets which influence cell and resultant plant size are deregulated independently of SA in these mutants.

NPR1 has been shown to function downstream of SA accumulation in SA-mediated expression of PR genes and SAR (Delaney et al., 1995; Cao et al., 1997; Shah et al., 1999). In our studies, double npr1-1/mpk4 mutants retain the dwarf, enhanced resistance and constitutive PR1 gene expression phenotypes of mpk4, but also exhibit the SA hypersensitivity typical of npr1-1. This suggests that mpk4 deregulates SA-mediated defenses independently of NPR1. This is consistent with the absence of the G-box, but presence of the W-box (TTGACT) and the 8-mer GACTWWHC motif, in 5′ upstream regions of constitutively expressed genes in mpk4. While these three motifs are regulatory elements in the PR1 promoter, the G-box may be specifically involved in positive regulation of PR1 by bZip factors interacting with NPR1 (Zhang et al., 1999). Several other genes affecting pathogen responses and SAR also act independently of, or partially through, NPR1. These include CPR6 (Clarke et al., 1998), ACD6 (Rate et al., 1999), CPR5 (Bowling et al., 1997) and the npr1 suppressor SSI1 (Shah et al., 1999). In cpr5 and in Isd mutants (lesions simulating disease resistance response; Dietrich et al., 1997), SAR is accompanied by the formation of spontaneous necrotic lesions. Since lesions are not observed in mpk4, MPK4 exerts a function downstream of the HR in SAR development. Examining the epistatic relationship of mpk4 to SA-regulatory mutants such as eds1 and pad4 (Falk et al., 1999; Jirage et al., 1999) may help determine the hierarchy of these components in plant resistance.

Evidence from previous studies suggests that plant MAPKs participate in the integration of signals arising from diverse stress stimuli. For example, tobacco SIPK (SA induced protein kinase) is activated by the tobacco mosaic virus (TMV)/resistance gene N interaction, fungal elicitors, nitric oxide, SA and wounding (Romeis et al., 1999; Kumar and Klessig, 2000). Tobacco WIPK (wound induced protein kinase), is transcriptionally induced and activated by both wounding and the TMV/N-interaction (Seo et al., 1995; Zhang and Klessig, 1998). Interestingly, overexpression of WIPK leads to elevated JA levels and constitutive expression of the JA-responsive gene PI-II (Seo et al. 1999). Furthermore, while wild type plants accumulate JA and its target gene mRNAs in response to wounding, sense-suppressed wipk plants accumulate SA and express SAR target genes (Seo et al., 1995). Our microarray and RNA blot analyses show that induction of certain JA-responsive genes is blocked in mpk4. This effect is independent of SA levels as mpk4 mutants expressing nahG also fail to accumulate PDF1.2 and THI2.1 mRNA in response to MeJA.

Thus, in addition to repressing SA-mediated defenses, MPK4 is required for JA-mediated gene expression. MPK4 may therefore be involved in integrating SA- or JA-dependent responses to selectively engage defenses against particular pathogen types or environmental stresses (Pieterse and van Loon, 1999; Felton et al., 1999).

### REFERENCES

Agrios, 1988, Plant Pathology, 3re ed. Academic Press, Inc., New York

Alonso et al., 1999, Science 284, 2148-2152.

Altschul et al., 1990, J. Mol. Biol. 215, 403-410.

Alvarez et al., 1998, Cell 92, 773-784.

Aoyama & Chua, 1997, Plant Journal 11, 605-612.

Artus et al., 1996, Proc. Natl. Acad. Sci. 93, 13404-09.

Bardwell et al., 1998, Genes and Development 12, 2887-2898.

Bowling et al., 1994, Plant Cell 6, 1845-1857.

Bowling et al., 1997, Plant Cell 9, 1573-1584.

Braam et al., 1990, Cell 60, 357-364.

Caddick et al., 1998, Nature Biotechnology 16, 177-180.

Cao et al., 1994, Plant Cell. 6, 1583-1592.

Cao et al., 1997, Cell 88, 57-63.

Christensen & Quail, 1996, Transgenic Research 5,213-218.

Clarke et al., 1998, Plant Cell 10, 557-569.

Clough et al., 2000, Proc. Natl. Acad. Sci. USA 97, 9323-9328.

Delaney et al., 1995, Proc. Natl. Acad. Sci. USA 92, 6602-6606.

Dietrich et al., 1997, Cell 88, 685-694.

Eulgem et al., 1999, EMBO J. 18, 4689-4699.

Falk et al., 1999, Proc. Natl Acad. Sci. USA 96, 3292-3297.

Feldman et al., 1989, Science 243, 1351-4.

Felton et al., 1999, Curr. Biol. 9, 317-320.

Forsburg and Sherman, 1997, Gene 191, 191-195.

Gaffney et al, 1993, Science 261, 754-756.

Glazebrook et al., 1997, Annu. Rev. Genet 31, 547-569.

Glazebrook, 1999, Plant Biol. 2, 280-286.

Grantz et al., 1995, Plant Physiol. 108, 411-418.

Gray et al., 1998, Proc. Natl. Acad. Sci. 95, 7197-7202.

Greenberg et al., 1994, Cell 77, 551-563.

Gupta et al., 2000, Mol. Plant-Microbe Interact. 13, 503-511.

Hua & Meyerowitz, 1998, Cell 94, 261-271.

Huang et al., 2000, Plant Physiology 122, 1301-1310.

Huang & Miller, 1991, Adv. Appl. Math. 12,337-357.

Ichimura et al., 1998, Biochem. Biophys. Res. Commun. 253, 532-43.

Ishige-Fumiharu et al., 1999, Plant Journal 18, 443-448.

Jach et al., 1995, Plant Journal 8, 97-109.

Jelitto-Van Dooren et al., 1999, Plant Cell 11, 1935-1944.

Jirage et al., 1999, Proc. Natl. Acad. Sci. USA 96, 13583-13588.

Johansen, 1997, Ann. Bot. 80, 697-700.

Krysan et al., 1996, Proc. Natl. Acad. Sci. 93, 8145-50.

Kumar and Klessig, 2000, Mol. Plant Microbe Interact 13, 347-351.

Last et al., 1991, Theoretical and applied genetics 81, 581-588.

Lawrence et al., 1993, Alignment. Science 262, 208-214.

Lebel et al., 1998, Plant J. 16, 223-233.

Leung et al., 1994, Science 264, 1448-1452.

Li & Chory, 1997, J. Cell 90, 929-938.

Li et al., 1999, Cell. 98, 329-339.

Ligterink et al., 1997, Science 276, 2054-57.

Liu et al., 1995, Plant J. 8, 457-63.

Madhani & Fink, 1998, Trends in Genet 14, 151-155.

Malamy et al., 1990, Science 250, 1002-1004.

Maniatis et al., 1982, Molecular Cloning: A laboratory Manual.

Martinez et al., 1999, Plant Journal 19, 97-106.

McElroy et al., 1990, Plant Cell 2, 163-172.

Merkouropoulos et al., 1999, Planta 208, 212-219.

Mett et al., 1993, Proc. Natl. Acad. Sci. 90, 4567-4571.

Miyata et al., 1999, Biochimica et Biophysica Acta, 1451, 334-342.

Miklos & Rubin, 1996, Cell 86, 521-529.

Mittler et al., 1995, Plant Cell 7, 29-42.

Mizoguchi et al., 1993, FEBS Lett. 336, 440-444.

Mizoguchi et al., 1996, Proc. Natl. Acad. Sci. 93, 765-769.

Mizoguchi et al., 1997, TIBTECH 15, 15-19.

Molina and Olmedo, 1997, Plant J. 12, 669-675.

Molina et al., 1999, Plant J. 17, 667-678.

Morris et al., 1997, Plant Mol. Biol. 35, 1057-1064.

Mantyla et al., 1995, Plant Physiol. 107, 141-148.

Nawrath and Metraux, 1999, Plant Cell. 11, 1393-1404.

Parker et al., 1996, Plant Cell 8, 2033-46.

Parker et al., 1997, Plant Cell 9, 879-94.

Pearson & Lipman, 1988, Proc. Natl. Acad. Sci. 85, 2444-2448.

Peng et al., 1997, Genes & Development 11, 3194-3205.

Penninckx et al., 1996, Plant Cell 8, 2309-2323.

Petersen et al. 2000 Cell 103, p.1-10.

Pieterse and van Loon, 1999, Trends Plant Sci. 4,52-58.

Rate et al., 1999, Plant Cell 11, 1695-1708.

Robinson et al. 1998, Current Biology, 8,1141-1150

Romeis et al., 1999, Plant Cell 11, 273-87.

Ruan et al., 1998, Plant J. 15, 821-833.

Schenk et al., 2000, PNAS 97, 11655-11660.

Seibertz et al., 1989, Plant Cell 1, 961-968.

Seo et al. 1999, Plant Cell. 11, 289-98.

Shah et al., 1999, Plant Cell. 11, 191-206.

Somerville & Somerville, 1999, Science 285, 380-383.

Staskawicz et al., 1995, Science 268, 661-667.

Sundaresan et al., 1995, Genes Develop. 9, 1797-1810.

Taipalensuu et al., 1997, Eur. J. Biochem. 247, 963-971.

Takahashi & Komeda, 1989, Mol. Gen. Genet 219, 365-72.

Thomma et al., 1998, Proc. Natl. Acad. Sci. USA 95, 15107-15111.

Vogel et al., 1998, Proc. Natl. Acad. Sci. 5, 4766-4771.

Ward et al., 1991, Plant Cell 3, 1085-1094.

Weymann et al., 1995, Plant Cell 7, 2013-2022.

Whalen et al., 1991, Plant Cell 3, 49-60.

Xie et al., 1998, Science 280, 1091-1094.

Yang et al., 1997, Genes and Develop. 11, 1621-1639.

Zhang & Klessig, 1997, Plant Cell, 9, 809-824.

Zhang & Klessig, 1998, Proc. Natl. Acad. Sci. 95, 7433-38.

Zhang et al., 1995, Plant J. 8, 139-145.

Zhang et al., 1999, Proc. Natl. Acad. Sci. USA 96, 6523-6528.

Zhou et al., 1998, Plant Cell. 10, 1021-1030.

1. A method of controlling the growth of a plant and/or the expression of at least one wounding or pathogen response gene in said plant, the method comprising altering in the plant the level of the gene product of a MAPK4 gene.

2. A method according to claim 1, wherein the level of the gene product of a MAPK4 gene is altered in the plant by the following steps
a) providing a recombinant DNA construct in a suitable vector in which the coding region of a MAPK4 gene is operably linked in an anti-sense orientation to an appropriate promoter such that the expression of the MAPK4 gene is regulated by said promoter;
b) transforming regenerable cells of a plant with said recombinant DNA construct; and
c) regenerating a transgenic plant from said transformed cell.

3. A method according to claim 2 wherein the transformation with the antisense MAPK4 construct leads to an increased content of salicylic acid (SA) in the transgenic plant.

4. A method according to claim 3 wherein the increased content of salicylic acid results in the induction of a SA-dependent systemic acquired resistance (SAR).

5. A method according to claim 2 wherein the DNA construct comprises a further MAPK4 gene which is operably linked to an appropriate promoter, such that the expression of that further MAPK4 gene is regulated by said promoter.

6. A method according to claim 5 wherein the further MAPK4 gene is overexpressed.

7. A method according to claim 5 wherein the product of the further MAPK4 gene is produced in constitutively active form.

8. A method according to claim 6 or 7 wherein the increased expression and/or activity of MAPK4 leads to an increased response to jasmonates (JAs) in the transgenic plant.

9. A method according to claim 8 wherein the increased JA-response results in the expression of JA-responsive genes selected from the group consisting of PDF1.2 and THI2.1.

10. A method according to claim 1, wherein the level of the gene product of a MAPK4 gene is altered in the plant by the following steps
a) providing a gene coding for an active MAPKK,
b) fusing said MAPKK gene with a recombinant DNA construct in a suitable vector in which the coding region of a MAPK4 gene is operably linked to an appropriate promoter such that the expression of the MAPK4 gene is regulated by said promoter, in order to obtain an activated MAPKK/MAPK4 fusion protein,
c) transforming regenerable cells of a plant with said constitutively activated MAPK4,
d) regenerating a transgenic plant from said transformed cell.

11. A method according to claim 10 wherein the expression of the MAPK4 gene is increased relative to the wild type gene.

12. A method according to claim 11 wherein the increased expression of MAPK4 leads to an increased response to jasmonates (JAs) in the transgenic plant.

13. A method according to claim 12 wherein the increased JAs-response results in the expression of JAs-responsive pathogen genes selected from the group consisting of PDF1.2 and THI2.1.

14. A method according to claim 1, wherein the level of the gene product of a MAPK4 gene is altered in the plant by the following steps
a) providing a recombinant DNA construct in a suitable vector in which the coding region of a gene coding for a catalytically inactive MAPK4 is operably linked in a sense orientation to an appropriate promoter such that the expression of the catalytically inactive MAPK4 gene is regulated by said promoter;
b) transforming regenerable cells of a plant with said recombinant DNA construct; and
c) regenerating a transgenic plant from said transformed cell.

15. A method according to claim 14 wherein the product of the MAPK4 gene cannot be phosphorylated.

16. A method according to claim 14 wherein the product of the inactive MAPK4 gene is non-phosphorylatable.

17. A method according to any of claims 2 to 16 wherein the promoter is a constitutive promoter.

18. A method according to claim 17 wherein the constitutive promoter is selected from the group consisting of cauliflower mosaic virus 35S promoter, cauliflower mosaic virus 90 with G-box 10 tetramer promoter, maize Adh promoter, maize ubiquitin Ubi-I promoter and rice Act1 promoter.

19. A method according to any of claims 2 to 16 wherein the promoter is an inducible promoter.

20. A method according to claim 19 wherein the inducible promoter is selected from the group consisting the tetracycline repressor/operator controlled promoter, ecdysone agonist inducible promoter, glucocorticoid agonist inducible promoter, copper inducible promoter, ethanol inducible promoter, and tobacco wun 1 promoter.

21. A method according to claim 1, wherein the level of the gene product of a MAPK4 gene is altered in the plant by the following steps
a) mutating in a regenerable plant cell the MAPK4 gene so as to obtain a loss of function of said gene, and
b) regenerating a transgenic plant from said transformed cell.

22. A method according to claim 21 wherein the mutation is provided by inserting an insertion element within the MAPK4 gene.

23. A method according to claim 22 wherein the insertion element is selected from the group consisting of a T-DNA and a transposon.

24. A method according to any of claims 1 to 23 wherein the wounding or pathogen response gene is a gene coding for a gene product selected from the group consisting of chitinase, extensin (EXT1), β-1,3-glucanase (BGL2/PR2), β-1,3-glucanase (BGL3), glutathione S-transferase (ERD11), glutathione S-transferase (PM24), monodehydroascorbate reductase, pectin methylesterase (PME1), a lipid transfer protein (MTE17.7), a LRR receptor kinase, hypothetical protein, LRR-receptor kinase, oxalate oxidase-like (GLP5), a proline-rich protein and a hypothetical protein.

25. A method according to any of claims 1 to 24 wherein the wounding or pathogen response gene is overexpressed.

26. A method according to any of claims 1 to 25 wherein the overexpression results in an enhanced resistance to plant pathogens selected from the group consisting of viruses, fungi, bacteria, insects and nematodes.

27. A method according to any of claims 1 to 26 wherein the transgenic plant, relative to a wild type plant, has a reduced growth.

28. A method according to any of claims 1 to 27 wherein the plant is a monocot or a dicot.

29. A method according to any of claims 1 to 28 wherein the MAPK4 is AtMPK4 derived from Arabidopsis thaliana.

30. A transgenic plant having enhanced wound and/or disease resistance, said plant comprising an antisense MAPK4 construct, wherein said construct leads to an increase in the expression of wounding and/or pathogen responsive genes.

31. A transgenic plant having enhanced wound and/or disease resistance, said plant comprising a constitutively active form of MAPK4.

32. A transgenic plant having enhanced wound and/or disease resistance, said plant comprising a catalytically inactive MAPK4 construct, wherein said construct leads to an increase in the expression of wounding and/or pathogen responsive genes.

33. A transgenic plant having enhanced wound and/or disease resistance, said plant comprising a mutation in the MAPK4 gene which results in a loss of function of said gene, wherein said mutation leads to an increase in the expression of wounding and/or pathogen responsive genes.

34. A transgenic plant according to any of claims 30 to 33 wherein the plant, relative to the wild type plant, has reduced growth.

35. A transgenic plant according to any of claims 30 to 34 wherein the plant is a monocot or a dicot.

36. A recombinant DNA construct comprising the coding region of MAPK4 gene operably linked in an antisense orientation to an appropriate promoter.

37. A transgenic plant cell transformed with the DNA construct of claim 36.

38. A method of screening a plant population for plants carrying an insertion element within the MAPK4 gene whereby the gene is functionally inactivated, the method comprising the steps of
a) providing a MAPK4 specific primer and an insertion element specific primer,
b) providing DNA of each of said plants,
c) performing PCR reactions using said primers, and
d) selecting a plant carrying an insertion element within the MAPK4 gene whereby the gene is functionally inactivated by identifying a PCR product primed by said primers.

39. Use of a MAPK4 gene for providing MAPK4 primers useful in the method according to claim 38.
